(19) United States
(12) Patent Application Publication
(10) Pub. No.: US 2016/0361443 A1
(43) Pub. Date: Dec. 15, 2016

(54) Ultrasound Molecular Imaging With Targeted Biocompatible Microbubbles

(71) Applicant: University of Pittsburgh - Of the Commonwealth System of Higher Education, Pittsburgh, PA (US)

(72) Inventors: Flordeliza Villanueva, Pittsburgh, PA (US); Bin Qin, Pittsburgh, PA (US); Xucai Chen, Glenshaw, PA (US)

(21) Appl. No.: 15/179,138

(22) Filed: Jun. 10, 2016

Related U.S. Application Data
(60) Provisional application No. 62/175,162, filed on Jun. 12, 2015.

(51) Int. Cl. A61K 49/00

(52) U.S. Cl. CPC A61K 49/0091

(57) ABSTRACT

The present invention is related to cardiovascular contrast agents. In particular, compositions and methods for ultrasound cardiovascular contrast agents useful for molecular imaging and/or diagnosis of cardiovascular diseases and disorders. For example, cardiovascular disorders comprising ischemia and/or myocardial injury may be imaged and diagnoses by the present invention.

### STATEMENT OF GOVERNMENT INTEREST

This invention was made with government support under grant number HL077534 awarded by the National Institutes of Health. The government has certain rights in the invention.

### FIELD OF THE INVENTION

The present invention is related to cardiovascular contrast agents. In particular, compositions and methods for ultrasound cardiovascular contrast agents useful for molecular imaging and/or diagnosis of cardiovascular diseases and disorders. For example, cardiovascular disorders comprising ischemia and/or myocardial injury may be imaged and diagnoses by the present invention.

### BACKGROUND

The diagnosis of acute coronary syndrome (ACS) in patients presenting to the emergency department with a history of recent or ongoing chest pain can be a challenge, particularly in patients presenting with atypical symptoms and signs. True acute myocardial ischemia is associated with upregulation of leukocyte adhesion molecules, such as P-selectin, E-selectin, and intracellular adhesion molecule 1 (ICAM-1), which hence provide a molecular signature, or tissue memory, of a recent ischemic event.¹ FIG. 5.

Ultrasound molecular imaging (UMI) to identify overexpression of leukocyte adhesion molecules may offer an approach to the detection of recent myocardial ischemia². This technique relies on ultrasound detection of disease specific endothelial epitopes using gas-filled microbubbles (MBs) as probes that bind specifically to the epitopes via targeting moieties on the surface of MBs. Previously, the concept of echocardiographic ischemic memory imaging was demonstrated using MBs targeted to P-selectin via the naturally occurring tetrasaccharide ligand sialyl. Lewis X (sLeX).³

Because such MBs are not ideal for clinical use due to the complex carbohydrate chemistry and possible immunogenicity the art is in need of an ischemic memory UMI probe that could be more readily extended to humans ⁴⁻⁶. In particular, the art is in need of a proven safe targeted double-layer microbubble^{7,8}.

### SUMMARY OF THE INVENTION

The present invention is related to cardiovascular contrast agents. In particular, compositions and methods for ultrasound cardiovascular contrast agents useful for molecular imaging and/or diagnosis of cardiovascular diseases and disorders. For example, cardiovascular disorders comprising ischemia and/or myocardial injury may be imaged and diagnoses by the present invention.

In one embodiment, the present invention contemplates a composition comprising a dual shell polymer microbubble conjugated to a peptide having a selective affinity for an adhesion molecule. In one embodiment, the adhesion molecule includes, but is not limited to E-selectin, P-selectin, ICAM, NCAM and/or VCAM. In one embodiment, the microbubble comprises an outer layer comprising a biologically compatible material. In one embodiment, the biologically compatible material is an amphiphlic material. In one embodiment, the microbubble comprises an inner layer comprising a biodegradable polymer. In one embodiment, the biodegradable polymer is a synthetic polymer. In one embodiment, the microbubble comprises a hollow core. In one embodiment, the hollow core encapsulates an acoustically active gas.

In one embodiment, the present invention contemplates a method comprising; a) providing: i) a pharmaceutically acceptable formulation comprising a dual shell polymer microbubble conjugated to a peptide having selective affinity for an adhesion molecule; and ii) an ultrasound imaging device; b) administering the formulation to a patient comprising at least one tissue overexpressing said adhesion molecule; c) imaging said at least one tissue with the ultrasound imaging device; d) measuring retention of said microbubble by said at least one tissue; and e) diagnosing the patient with a medical condition when the patient microbubble retention is greater than a control microbubble retention. In one embodiment, the at least one tissue is a cardiovascular tissue. In one embodiment, the medical condition is a cardiovascular disease. In one embodiment, the cardiovascular disease comprises a myocardial infarction.

### DEFINITIONS

The term “polymer contrast microbubble” as used herein, refers to any approximately spherical structure comprising at least one polymer having a diameter of approximately 100 nanometer to 10 microns. Microbubbles may comprise a single layer surface or a bi-layer surface. In conventional usage in clinical ultrasound imaging, the compositions of these microbubbles result in a harmonic oscillation to certain ultrasound frequencies, such that their emissions may be visualized as an image. Alteration of polymer characteristics including, but not limited to, polymer type or concentration, can modify the structural and functional characteristics of a microbubble. Polymers may comprise a linked series of individual components, wherein the components may include, but are not limited to, small organic molecules, amino acids, or nucleic acids. For example, a polymer as contemplated herein includes a peptide or protein and/or an oligonucleotide. A microbubble may contain single or multiple pockets which hold a gas (i.e., for example, an acoustically active gas) or a liquid (i.e., for example, an acoustically active liquid, for example those by Wickline). In general, microbubbles are roughly spherical or irregular shape and have at least one continuous phase comprising a polymer as defined herein. Further, the continuous phase may form one or multiple enclosures capable of entrapping a gas or liquid (i.e., for example, gases or liquids that are acoustically active). Microbubbles may be fabricated by various methods including, but not limited to, double emulsion evaporation or extraction, single emulsion evaporation or extraction, spray drying, freeze spray drying, ultrasound sonication, or microfluidics.

The term “bi-layer microbubble” or “dual shell microbubble” as used herein, refers to any microbubble comprising a shell having an inner and outer layer. Generally, the outer layer comprises ligands that are amphiphilic and biocompatible (i.e., for example, albumin) and the inner layer comprises biodegradable polymers. The bi-layer is usually between approximately 25-750 nm in width.

The term “controlled fragility microbubble” as used herein, refers to microbubbles within a population of microbubbles having the same wall thickness to diameter ratio.

The term “microparticles” as used herein, refer to solid particles ranging from 100 nm to 10 microns in diameter which lack pores such that a gas or liquid is unable to become embedded in the solid continuous phase. Microparticles may be fabricated by various methods including, but not limited to, double emulsion evaporation or extraction, single emulsion evaporation or extraction, spray drying, freeze spray drying, ultrasound sonication, or microfluidics.

The term “target tissue' as used herein, refers to any cellular structure having biological functionality including, but not limited to, endocytosis and/or extracellular ligand binding sites that may facilitate interaction with a microbubble. A target tissue may comprise a biological cell that may be naturally occurring or synthetic and is preferably viable. Such biological cells may be a stem cell including, but not limited to, bone marrow-derived stem cells, embryonic stem cells, adult stem cells, hemopoeitic stem cells, mesenchymal stem cells, epidermal stem cells, endothelial stem cells, endothelial progenitor cells, resident cardiac stem cells, pluripotent stem cells, adipose-derived stem cells, cancer stem cells (i.e., for example, a leukemic hemopoeitic stem cell) or skeletal myoblasts. Alternatively, biological cells may include, but are not limited to, brain cells, liver cells, muscle cells, nerve cells, chondrocytes, lymphocytes, intestinal cells, pancreatic cells, liver cells, heart cells, lung cells, colon cells, bladder cells, uterine cells, prostate cells, urethra cells, testicular cells, and/or epithelial cells. Further, a biological cell may be a cancerous cell, for example, a dendritic cell.

The term “contrast agent” as used herein refers to any composition capable of improving the intensity discrimination between at least two different structures having differential densities. Contrast agents contemplated herein include, but are not limited to, polymer microbubbles, stabilized microbubbles, sonicated albumin, gas-filled microspheres, gas-filled liposomes, and gas-forming emulsions.

The term “anatomical site” as used herein, refers to any location within a biological organism comprising a target tissue, target organ, and/or a target plurality of cells.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences. Other concerns are the adverse reaction of the patient to conventional, iodine based contrast agents.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “imaging” as used herein, refers to any technique wherein a visual representation of a biological cell is created. Such imaging may occur either in vitro, in situ, or in vivo. With specific reference to in vivo imaging, the resolution of the visual representations may be enhanced when performed in combination with a contrast agent.

The term “tracking” as used herein, refers to monitoring the migration of a composition from one physical location to another physical location by using imaging techniques. For example, a biological cell comprising internalized microbubbles may be monitored during a migration from a first anatomical site to a second anatomical site.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc. Another type of injury is the adverse reaction of a patient to conventional contrast agents containing iodine.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “administered” or “administering” as used herein, refers to any method of providing a composition (i.e., for example, a biological cell comprising an internalized microbubble) to a patient such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a compound or sequence. In one respect, a compound or sequence may be derived from an organism or particular species. In another respect, a compound or sequence may be derived from a larger complex or sequence. The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier” or “pharmaceutically acceptable vehicle”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules that are removed from their natural environment, isolated or separated, and are at least 60% free, preferably 75% free, and more preferably 90% free from other components with which they are naturally associated. The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

### BRIEF DESCRIPTION OF THE FIGURES

FIG. 1 provides one embodiment of peptide-microbubble conjugation using maleimide chemistry.

FIG. 2 presents exemplary data showing the effect of peptide concentration on thiol-maleimide reaction and microbubble/peptide non-specific binding.

FIG. 3 presents exemplary data showing the effect of incubation time on thiol-maleimide reaction and nonspecific binding on polymer microbubbles.

FIG. 4 presents exemplary data using an in vitro flow chamber showing the number of polymer microbubbles bound to activated or non-activated HUVEC cells after shear force application.

FIG. 5 illustrates some embodiments of leukocyte adhesion molecules.

FIG. 6 illustrates an in vitro flow chamber in which some studies discussed herein were performed.

FIG. 7 depicts one embodiment of a targeted microbubble comprising a biotin-streptavidin linker.

FIG. 8 illustrates an in vitro parallel perfusion plate for performance of cell shear force stability studies.

FIG. 9 presents the results of a cell shear force stability study.

FIG. 9A: Photomicrographs of E-selectin targeted microbubbles and control microbubbles.

FIG. 9B: Graphical presentation of the data observed in FIG. 9A.

FIG. 10 presents the results of an intravital microscopy study.

FIG. 10A: Photomicrographs of E-selectin targeted microbubbles and control microbubbles.

FIG. 10B: Graphical presentation of the data observed in FIG. 10A.

FIG. 11 presents photomicrographs before and after ischemic reperfusion injury.

FIG. 12 presents exemplary data showing E-selectin targeted microbubble binding to tissues subjected to ischemic reperfusion injury.

FIG. 13 presents exemplary data showing ICAM-1 and E-selectin expression during ischemia-reperfusion obtained by quantitative real-time polymerase chain reaction.

FIG. 14 presents several embodiments of E-selectin targeting peptides.

FIG. 15 presents one embodiment of a maleimide linker synthetic pathway.

FIG. 16 presents exemplary data showing microbubble adhesion to rat microvascular endothelial cells (ECs) in vitro. *p<0.02 for MBESEL in inflamed ECs versus noninflamed ECs. {p<0.02 for MBESEL versus MBCTL in inflamed ECs. {p<0.01 for MBESEL versus MBIgG in inflamed ECs.

FIG. 17 presents exemplary data showing in vivo adherence of E-selectin targeted and control microbubbles (MBs) to rat cremaster microcirculation. *p<0.04 MBESEL in inflamed versus noninflamed microcirculation. {p<0.04 MBESEL versus MBCTL in inflamed microcirculation. {p=0.03 MBESEL versus MBIgG in inflamed microcirculation.

FIG. 18 presents exemplary data of fluorescent micrographs of rat cremaster microcirculation. Fluorescent microbubble attachment under TNF-α induced inflammation (A, B, C) or basal conditions (D, E, F) after intravenous MBESEL (A, D), MBCTL (B, E), and MBIgG (C, F). After MBESEL injection, there was adhesion to inflamed circulation (arrows) but not to control, noninflamed circulation. There was minimal adhesion of the control microbubbles to normal or inflamed circulation.

FIG. 19 presents exemplary data of background-subtracted, color coded ultrasound contrast images of the left ventricle in the short-axis view during coronary occlusion (A) and 4 hours after reperfusion (B, D to F). A, During coronary occlusion and injection of nontargeted microbubbles, there was a risk area (region between the arrows), corresponding to the left anterior descending (LAD) coronary artery territory. B, During reperfusion and injection of nontargeted microbubbles, there was homogeneous contrast enhancement of the myocardium. C, There was no infarction by triphenyl tetrazolium chloride staining. D, After injection of MBESEL, there was persistent contrast enhancement of the previously ischemic LAD coronary artery territory, not seen after injection of either of the MBCTL (E) or MBIgG (F).

FIG. 20 presents exemplary data showing videointensity measurements in the postischemic and nonischemic regions after injection of three microbubble types. *p<0.02 MBESEL in ischemic bed versus nonischemic bed. {p<0.03 MBESEL versus MBCTL in ischemic bed. {p+0.03 MBESEL versus MBIgG in ischemic bed.

FIG. 21 presents exemplary data showing immunofluorescent staining of postischemic (A-C) and nonischemic (D-F) myocardium 4 hours after reperfusion. Nuclei were stained blue with 49,6-diamidino-2-phenylindole, E selectin was stained red with CY3, and ICAM-1 was stained green with FITC. Nonspecific IgG was a control stain.

FIG. 22 presents examplary of microbubble design results.

FIG. 22A: Size distribution of polymer microbubbles before and after peptide conjugation. Peptide conjugation does not change the microbubble size distribution.

FIG. 22B: Fluorescence microscopic images of polymer microbubbles before and after coupling with FITC-labelled E-selectin peptide (Insert in corners: bright field images of MBs).

FIG. 23 presents exemplary data showing the quantitation of peptide conjugation to a microbubble population.

FIG. 23A. Comparison of peptide amount attached on MB using maleimide/thiol chemistry and biotin/streptavidin linker (mean±SD, n=6, **: p<0.01; n.s.: not statistically significant). The amount of FITC-labeled peptide coupled to microbubble was quantified by flow cytometry, and calculated from a standard calibration line between fluorescence peak channel number and the number of molecules of equivalent fluorescein (MEFL) generated from 8-peak rainbow calibration particles (BD). The amount of peptide bonded with thioletherbond or biotin/streptavidin interaction was calculated by subtracting amount of nonspecifically absorbed peptide from the total amount of peptide attached to MB.

FIG. 23B: Stability of coupled peptide in mouse serum. The FITC-labeled peptide coupled MBs using maleimide chemistry was incubated in 50% mouse serum. Samples at different time point were analyzed by flow cytometry.

FIG. 24 presents exempary data of TNF-α induced E-selectin upregulation by immunostaining of HUVEC cells.

FIG. 24A: Absence of TNFα (NT);

FIG. 24B: Presence of TNFα (20 ng/ml).

FIG. 24C: Electrophoresis gel showing a significant upregulation of E-selectin after incubation with TNFα (20 ng/mL) for 4 hr as compared to E-selectin without TNFα (NT).

FIG. 25 presents exemplary microscopy images of microbubble Esel(MAL) binding to HUVEC cell monolayers using a parallel flow chamber.

FIG. 25A: Untreated HUVEC cells.

FIG. 25B: TNFα activated HUVEC cells.

FIG. 25C: Binding of various MBs to HUVEC cell monolayer were evaluated under flow condition (shear rate: 100 sec ⁻¹) using 1 mL medium containing 5×10⁶ MBs. Each data point represents a mean value of cell bound MB from 20 images randomly taken from each sample.

FIG. 26 presents exemplary data comparing myocardial retention of microbubble populations (2×10⁷) either with, or without, a targeting ligand in an LPS rat inflammation model. The data were linearized and a two-phase washout model was fitted to obtain the retained MB concentration in the myocardium.

FIG. 26A: Myocardial signal intensity following a control IgG MB population (control).

FIG. 26B: Myocardial signal intensity following an E-sel target MB (Mal) MB population.

### DETAILED DESCRIPTION OF THE INVENTION

The present invention is related to cardiovascular contrast agents. In particular, compositions and methods for ultrasound cardiovascular contrast agents useful for molecular imaging and/or diagnosis of cardiovascular diseases and disorders. For example, cardiovascular disorders comprising ischemia and/or myocardial injury may be imaged and diagnoses by the present invention.

A targeting peptide ligand has been developed that may be conjugated to a biocompatible microbubble for ultrasound molecular imaging of cardiovascular diseases. In one embodiment, the targeted microbubble can selectively adhere to inflamed endothelial cells through binding to E selectin, and thus allow ultrasound detection of myocardium ischemia. Conventional linking chemistries currently approved for human can be used for the proposed peptide conjugations discussed below.

Methods contemplated by this invention provide for a rapid bedside diagnosis of a cardiac etiology for chest pain in patients present to the emergency room generally presented as chest discomfort of unknown etiology. Patients with positive studies can be admitted to the hospital, and those with negative studies can either be sent home or evaluated for non-cardiac causes of chest pain. This invention therefore provides for point of care triage capabilities for Emergency room physicians. The use of a peptide, as opposed to an antibody, linked to a microbubble (e.g., for example, a polymer/albumin double shell microbubble) for which there is extensive safety data, places some embodiments of the present invention at the forefront of clinical applicability.

### I. Cardiovascular Imaging

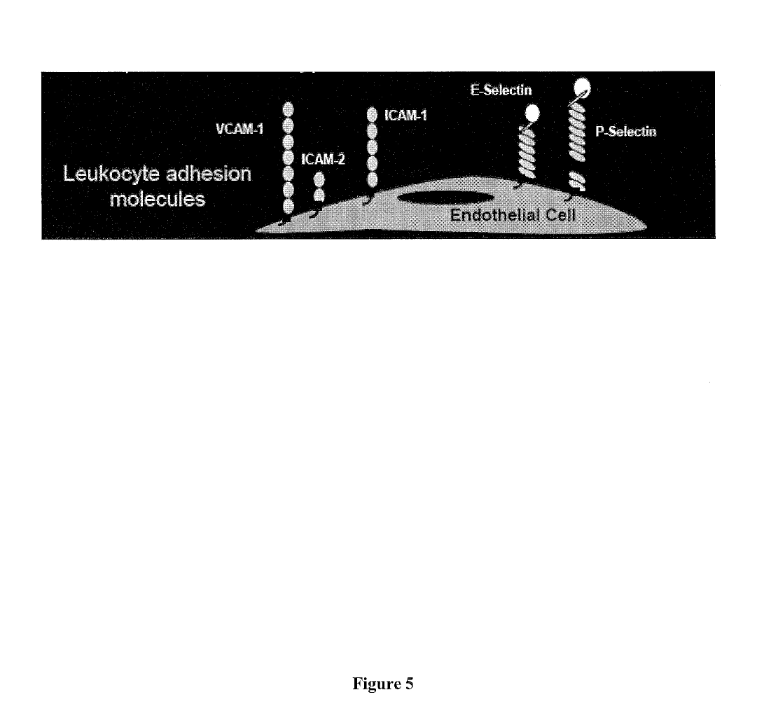

Detection of upregulated leukocyte adhesion molecules using antibody or carbohydrate ligands on microbubbles (MB) has proven the concept that recent myocardial ischemia can be identified with ultrasound molecular imaging. See, FIG. 5; and Collins et al., Trends Cardiovasc Med 1993;3. Clinical translation has been limited by immunogenicity or unfavorable chemistry of the targeting ligand.

Targeted microbubbles have been developed for ultimate human use in myocardial ischemic memory imaging. These E-selectin-targeted microbubbles made echo identification of recently ischemic myocardium possible via a short synthetic peptide. These data set the stage for clinical application of ultrasound ischemic memory imaging in the diagnosis of acute coronary syndrome in patients presenting with chest pain of uncertain etiology. Leng et al., “Detection of Recent Myocardial Ischemia Using a New Contrast Agent Designed for Human Application” Circulation 122:A20200 (2010).

### II. Conventional Microbubble Diagnostic Platforms

A disulfide linkage that conjugated a cyclic RGD pentapeptide lipid microbubble has been used for breast cancer imaging. Anderson et al., Invest Radiol. 2011; 46(4): 215-224.)

A lipid microbubble conjugated with a selectin ligand sialyl Lewis X targeted P-selectin to demonstrate feasibility of myocardial ischemic memory imaging in an animal model. Villanueva et al., Circulation 2007;115:345-352.

A lipid microbubble functionalized with heterodimer peptide has been targeted to VEGFR2 (e.g., BR55; Bracco Diagnostics Inc.). BR55 has been reported to be used for tumor angiogenesis imaging. For example, a Phase 0 clinical trial of BR55 for prostate cancer detection was completed in December 2012. Currently a Phase 1 clinical trial is recruiting subjects. Invest Radiol 2010;45: 89-95; clinicaltrials.gov/ct2/show/NCT01253213?term=BR55&rank=1; and clinicaltrials.gov/ct2/show/NCT02142608?term=BR55&rank=2.

A poly n-butyl cyanoacrylate microbubble has been reported that is covalently coupled to a short E selectin targeting peptide (IELLQAR; SEQ ID NO:2). This microbubble can be used to detect tumor vessels in nude mice for treatment of human ovarian carcinoma xenografts. Fokong et al., Invest Radiol 2013;48: 843-850)

A biotin-streptavidin conjugated rat monoclonal ICAM-1 antibody lipid microbubble was used to quantify vascular inflammatory change associated with atherosclerosis. Kaufmann et al., Circulation (2007) 116:276-284.

Other references also provide relevant information to the development and use of conjugated microbubbles. “Microparticles useful as ultrasonic contrast agents” U.S. Pat. No. 6,193,951; “Peptides and compounds that bind to elam-1” European Patent Number 0700301; “Methods devices and systems of preparing targeted microbubble shells” U.S. Patent Application Publication Number 2013/0101521; “Ultrasound Imaging with Targeted Microbubbles” United States Patent Application Publication Number 2010/0196284; “Targeted microbubble” WO 2013/028942; “Targeted micro-bubble probe for magnetic resonance imaging and blood pressure monitoring and preparation method thereof” Chinese Patent Number 101912622; “Microbubble compositions, and methods for preparing and using same” United States Patent Application Publication Number 2005/0260189; and “Optical imaging contrast agents and uses thereof” United States Patent Application Publication Number 2012/0244078. (all patents and patent application publications are herein incorporated by reference).

### III. Targeted Biocompatible Microbubbles

Microbubble targeting ligands contemplated by the present invention comprise high affinity peptides to endothelial adhesion molecules. In one embodiment, the endothelial adhesion molecule comprises E-selection. Although it is not necessary to understand the mechanism of an invention, it is believed that these endothelial adhesion molecules may be associated with cardiovascular conditions.

In one embodiment, the targeting ligands described herein may be immobilized onto a microbubble shell. For example, the immobilization may be accomplished using a poly (ethylene glycol) (PEG) spacer and/or a covalent linker comprising a disulfide bond and/or thioether bond.

In one embodiment, the present invention contemplates a method comprising a systemic administration of targeted ultrasound contrast agent microbubbles to a patient, wherein said microbubble comprises an adhesion molecule targeting ligand. In one embodiment, the patient is believed to exhibit adhesion molecule overexpression. In one embodiment, the method further comprises monitoring the patient with an ultrasound scan to determine the amount of microbubble vascular retention. The method further comprises evaluating the amount of microbubble vascular retention to quantify adhesion molecule expression. In one embodiment, the amount of adhesion molecule expression diagnoses a cardiovascular disorder or other disease state. Although it is not necessary to understand the mechanism of an invention it is believed that the ultrasound detection of disease-specific endothelial receptors is accomplished by using microbubbles comprising targeting ligands that selectively bind to the target receptor.

A. Microbubble Composition And Preparation

In one embodiment, the present invention contemplates a contrast agent comprising a stabilized gas filled polymer/albumin double shelled microbubble. U.S. Pat. No. 6,193,951 (herein incorporated by reference). In one embodiment, the stabilized gas filled polymer/albumin doubles shelled microbubble further comprises a targeting ligand. In one embodiment, the targeting ligand is conjugated to the microbubble wall.

Usually microbubbles contemplated herein comprise a diameter range ranging between approximately 1-10 μm. Although it is not necessary to understand the mechanism of an invention it is believed that this size range restricts the microbubbles to circulate only within vascular compartments. In contrast to other conventional molecular imaging probes, ultrasound imaging using the microbubbles contemplated herein provide specific advantages for molecular imaging including, but not limited to, a lack of radioactivity exposure and/or a superior penetration depth as compared to chemical counterparts used for optical imaging. Another advantage of ultrasound with microbubbles as contemplated herein for molecular imaging is that these microbubbles are also capable of carrying therapeutics and rendering bio-effects under ultrasound, which makes combined imaging and therapy possible.

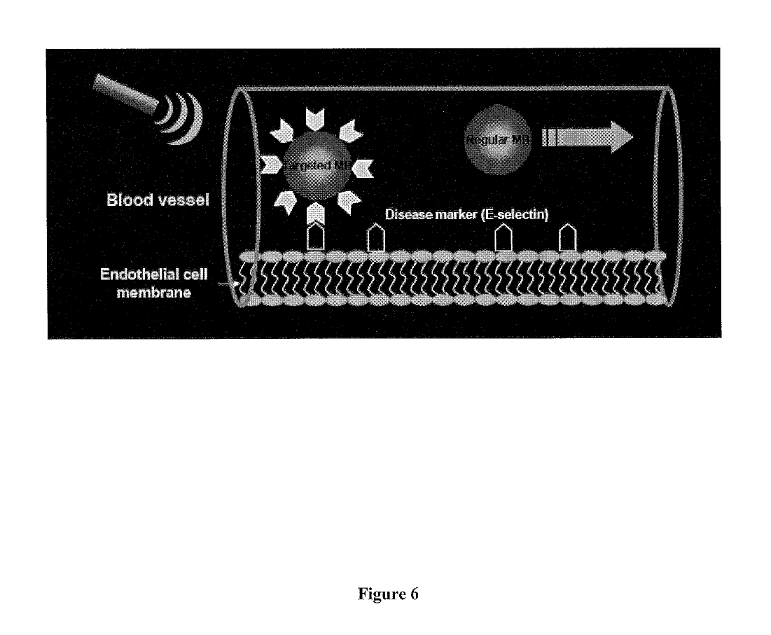

A biotin-streptavidin linker is convenient for ligand conjugation but not clinically translatable due to its potential immunogenicity; therefore a covalent linker such as thioether bond (maleimide-thiol chemistry) was used to conjugate peptides to polymer microbubbles for clinical applications. Using maleimide-thiol chemistry 18944±4308 peptides were conjugated per microbubble, which is similar to 22298±1962 peptide per microbubble using a biotin-streptavidin interaction. In vitro flow chamber studies confirmed that conjugating E selectin targeting peptide to microbubble using a thioether bond displayed did not alter cell adhesion of the microbubbles, which adhered to TNFα activated human endothelial HUVEC cells to a similar extent as did MBEsel (biotin-streptavidin linker). See, FIG. 6.

In one embodiment, the polymer microbubble (MB) has double layers: the inner layer comprises at least one polymer while the outer layer comprises a glutaraldehyde crosslinked human serum albumin (HSA). For example, each HSA has a plurality of disulfide bonds (e.g., for example, approximately seventeen) and at least one free thiol group, wherein the free thiol group may be use for peptide conjugation. Although amine groups could also potentially be used for conjugation, these moieties are not preferred as they have tendency to be modified during glutaraldehyde crosslinking

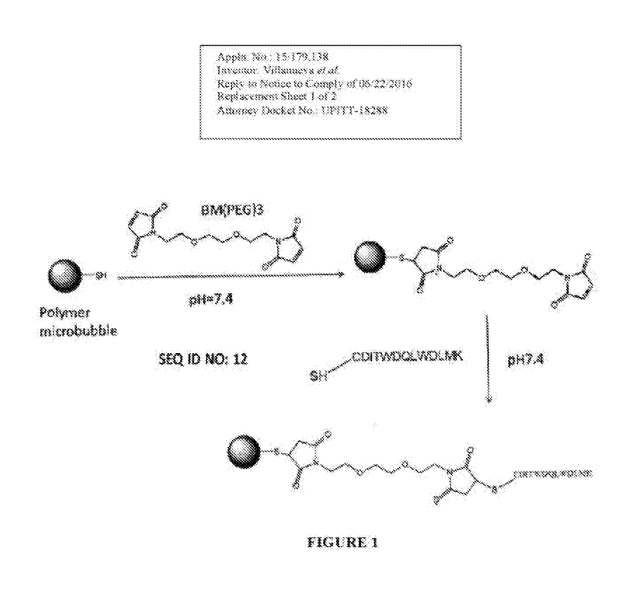

In one embodiment, the polymer microbubble may first react with an excess amount of BM(PEG)3 to generate a maleimide functional group, which can further react with E-selectin targeting peptide (DITWDQLWDLMK; SEQ ID NO:1) bearing one cysteine residue on the N-end. See, FIG. 1. Both steps involve a maleimide-thiol reaction and purification procedure. In theory, maleimide conjugation can be also performed in one step by mixing a polymer microbubble, BM(PEG)3, and a thiol peptide together; however, this process can potentially result in forming several byproducts, such as peptide dimers, and an overall low conjugation yield.

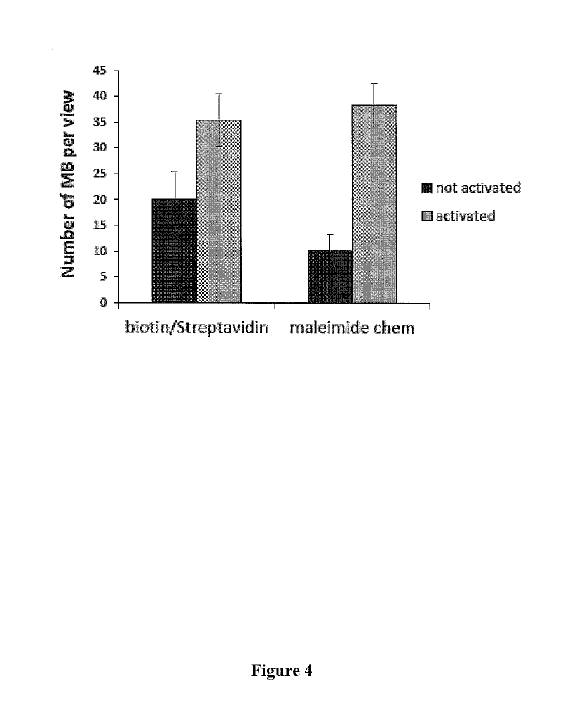

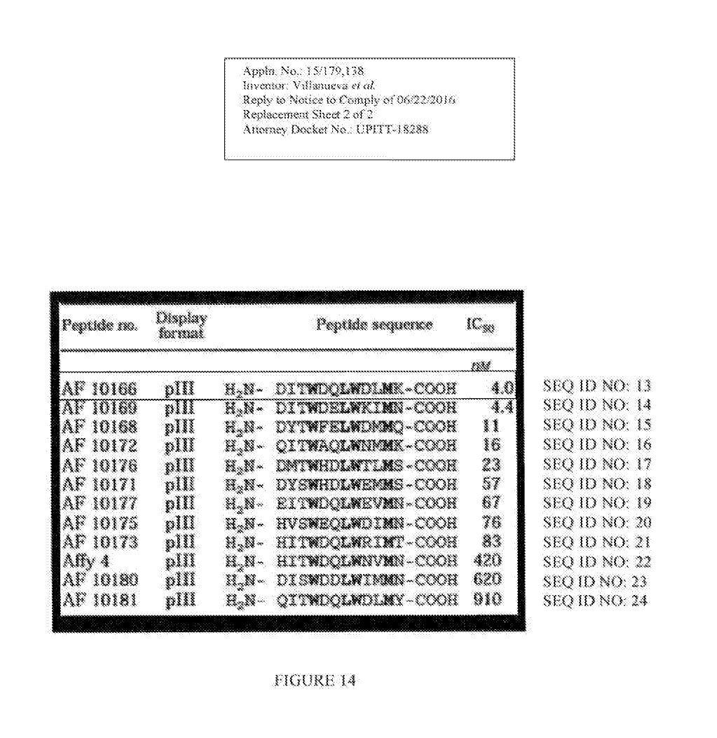

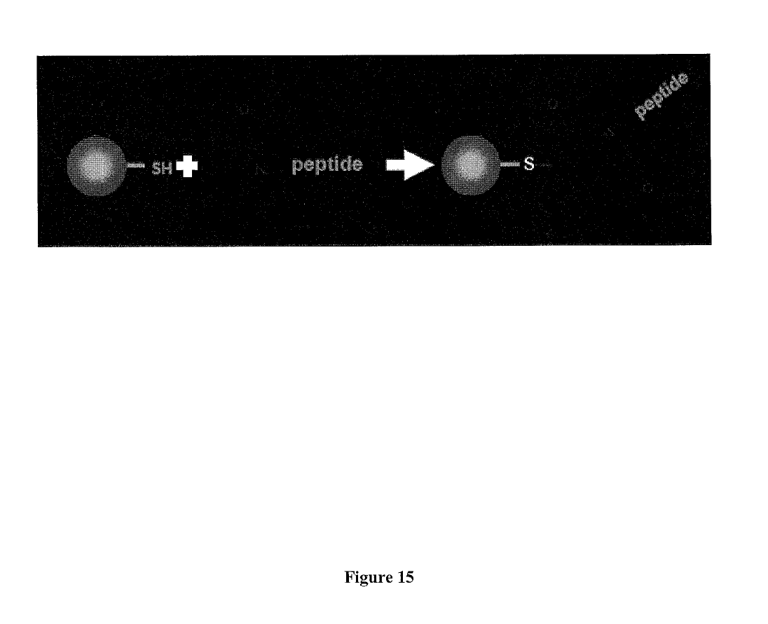

Although maleimide chemistry has been routinely used in organic synthesis, this presently disclosed synthetic scheme is designed based on chemistry specific for the presently disclosed polymer microbubble composition. See, FIG. 15. For example, in vitro flow chamber studies with non-activated and activated HUVECs demonstrated a substantial increase in adhesion of the targeted microbubbles on the activated cells. These data are comparable with commonly reported biotin-streptavidin linker techniques. See, FIG. 4.

In some embodiments, the present invention contemplates a contrast agent comprising a double-layer MB composed of an outer shell of crosslinked human albumin, an inner layer of biodegradable polymer (poly-DL-lactide [PDLLA]), and encapsulated nitrogen gas.⁹ The surface of the MBs may be coated with biotin for further targeting ligand attachment.¹⁰ For intravital microscopy, the MBs may be fluorescently labeled with Bodipy 493/503 (Life Technologies, Eugene, Oreg.). Biotinylated peptide AF10166 (H2NDITWDQLWDLMK-COOH, Peptides International), previously identified by phage display to have specific affinity to human E-selectin, was used as the targeting moiety.¹¹ The control ligands were either a biotinylated peptide composed of a random AF10166 sequence 12-mer (H2NWKLDTLDMIWQD-COOH) or rat biotinylated immunoglobulin G (IgG, BD Biosciences, San Diego, Calif.). The ligands were conjugated onto the MB via biotin-streptavidin bridging chemistry. MBs conjugated with AF10166 are henceforth designated as MBESEL, and MBs linked to the 12-mer randomized sequence or IgG are designated as MBCTL and MBIgG, respectively. MB diameter was 3.4 6 1.3 mm (Multisizer-III, Beckman Coulter, Beckman Coulter, Miami, Fla.).

B. Therapeutic Use Of Targeted Microbubbles

The data described herein provides results using an E-selectin targeting peptide-conjugated microbubble for ischemic memory detection (i.e. diagnosis of recent myocardial ischemia). The E selectin targeting peptide ligand (Accession Number AF10166; DITWDQLWDLMK (SEQ ID NO:1)) is believed to have a K_{d} value in the low nanomolar range, and has been previously developed using phage display library screening. E-selectin targeting peptides may comprise amino acid peptides discovered from phage display technology against human E-selectin. Martens, CL, et al., JBC, p21129-2136, 1995; FIG. 14. Such E-selectin targeting peptides have similar affinities for human, rat, and mouse E-selectin

In another embodiment, however, the method can also be applied to other disease entities associated with E selectin overexpression (inflammation). In some embodiments, the present invention facilitates acute coronary syndrome diagnosis in patients with recent or ongoing chest pain. Although it is not necessary to understand the mechanism of an invention, it is believed that that a primary symptom of recent/ongoing chest pain represents a significant diagnostic challenge in emergency room settings. For example, when true acute myocardial ischemia occurs, it is believed that there may be an upregulation of leukocyte adhesion proteins, which can serve as an inflammation marker for ischemic events. In one embodiment, E selectin comprises one such endothelial cell-specific adhesion glycoprotein associated with recent ischemia. Consequently, E selectin can serve as a molecular marker of recent ischemia, the detection of which may facilitate the diagnosis of chest pain as coming from an acute coronary syndrome. Various different linkage chemistries may be employed to conjugate an E selectin peptide ligand onto a polymer microbubble surface. For example, polymer microbubbles have been prepared bearing peptide ligands including, but not limited to: i) an E selectin targeting peptide; ii) a control peptide; and/or iii) a nonspecific IgG via biotin-streptavidin linker (denoted as MBEsel, MBcontrl or MBIgG).

Alternatively, adhesion of IL-6 targeted polymer microbubbles activated rat endothelial cells (EC) was evaluated using an in vitro flow chamber system. There was greater adhesion of MBEsel to activated EC than normal EC, whereas MBcontrl or MBIgG exhibited minimal adhesion to both activated and normal EC. Similarly, an E-selectin targeted polymer microbubble was monitored using echocardiography on rats 4 hrs after a transient coronary occlusion followed by reperfusion. Videointensity in postischemic myocardium after administration of MBEsel was higher than that of nonischemic bed and higher than that of MBcontrl or MBIgG, establishing the potential of this imaging strategy for detecting the presence and extent of recent myocardial ischemia.

C. In Vitro Parallel Plate Perfusion Study

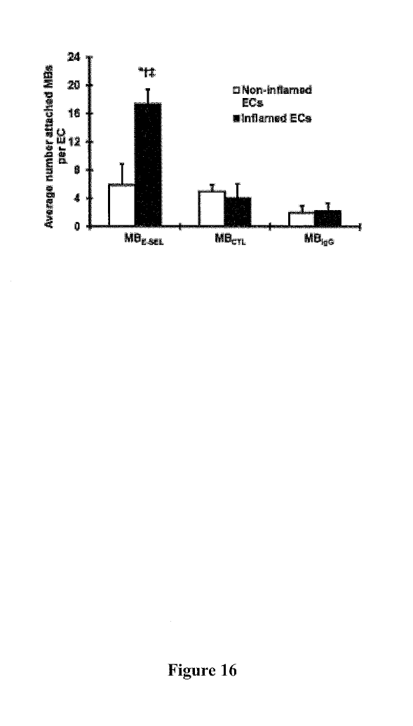

There was greater MB_{ESEL} adhesion to inflamed compared to noninflamed cells (17.4±1.8 vs 5.9±3.4 MB/EC, p<0.02). FIG. 16. There was greater adhesion of MB_{ESEL} to inflamed ECs (17.4±1.8 MB/EC) compared to MB_{CTL} (4.1±0.6 MB/EC, p<0.02) and MB_{IgG} (2.3±0.6 MB/EC, p<0.01). There was no difference in adhesion to activated versus normal ECs for MB_{CTL} (p=0.41) or MB_{IgG} (p=0.48).

D. Intravital Microscopy

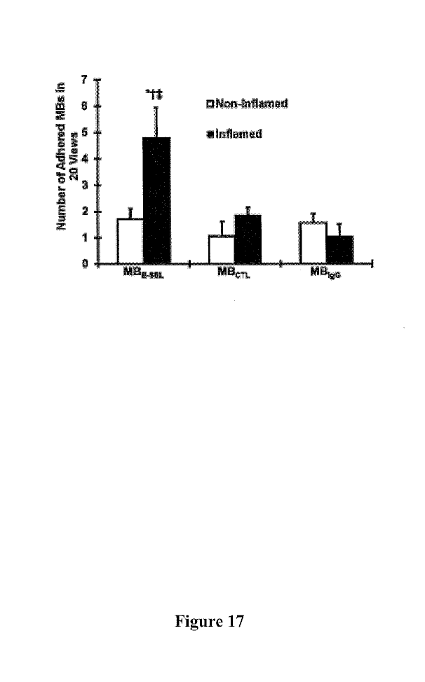

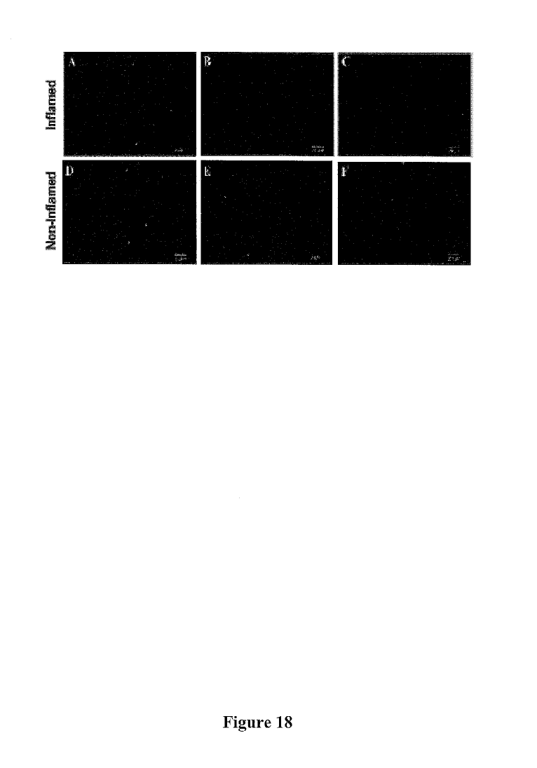

There was greater adhesion of MBESEL to inflamed microcirculation (4.8±1.2 MBs/field) compared to control MBIgG (1.1±0.3 MBs/field, p<0.03) and MBCTL (1.5±0.3 MBs/field, p<0.04) and compared to control, noninflamed microcirculation (1.7±0.4 MBs/field; p<0.04). FIG. 17. Fluorescence images of venular segments under basal (panels D to F) and TNF-a-activated (panels A to C) conditions are also illustrated. FIG. 18. There was attachment of Bodipy-labeled MBESEL under inflammatory conditions (FIG. 18A), which was less frequently seen under basal conditions (FIG. 18D). There was minimal attachment of MBCTL (FIGS. 18B and 18E) or MBIgG (FIGS. 18C and 18F) to normal or inflamed venules.

E. E-Selectin Targeted UMI

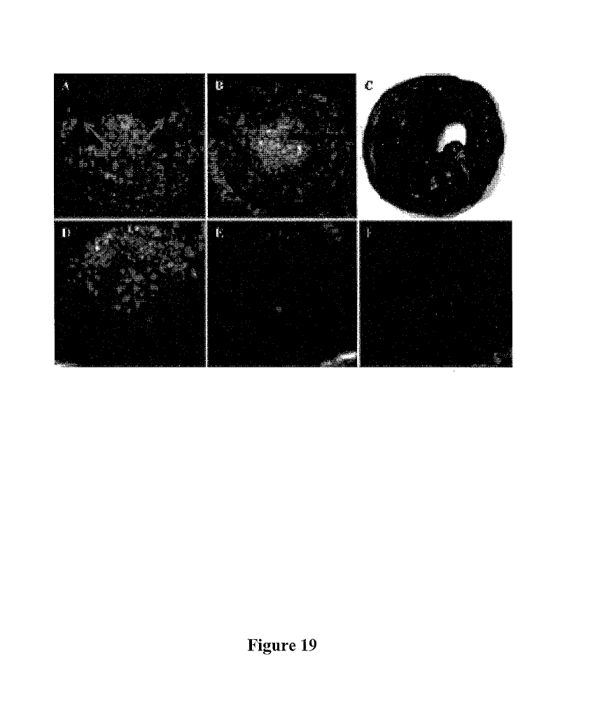

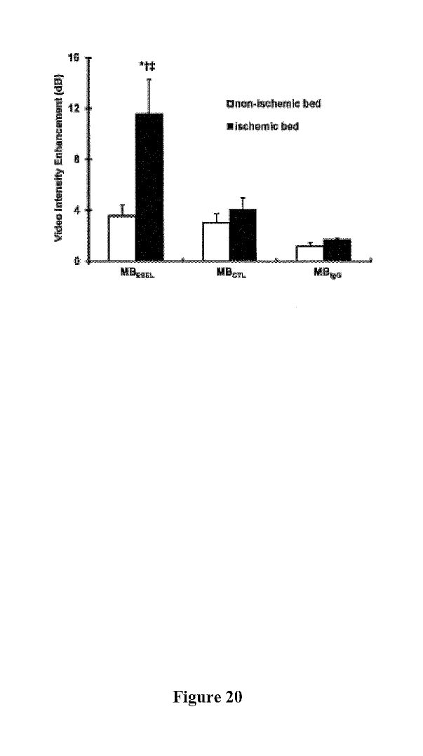

Background-subtracted color-coded contrast ultrasound images from a single rat are illustrated during occlusion and reperfusion. FIG. 19. During LAD coronary artery occlusion, injection of nontargeted MBs confirmed the presence of a risk area involving the anterior septum and anterior wall (see arrows, FIG. 19A). After release of the occlusion, nontargeted MCE showed resolution of the risk area (see FIG. 19B). FIG. 19D is a color-coded image 3 minutes after injection of MBESEL, after subtraction of the post-destruction frame. There was persistent contrast enhancement within a region that co-localized with the previously ischemic area LAD coronary artery territory, consistent with MBESEL binding to the postischemic bed. There was minimal persistent contrast enhancement after injection of either of the control MB species (FIGS. 19E and 19F). TTC staining confirmed the absence of infarction (see FIG. 19C). Background-subtracted VI data in the ischemic and nonischemic myocardium 3 minutes after MB injection are shown in FIG. 20. There was greater enhancement of the postischemic bed compared to the nonischemic bed after injection of the MBESEL (11.6±2.7 dB vs 3.6±0.8 dB; p<0.02). Furthermore, contrast enhancement of the LAD coronary artery bed was higher after injection of MBESEL compared to the control MBCTL (4.0±1.0 dB; p<0.03) and MBIgG (1.7±0.1 dB; p<0.03). A significant linear relationship was found between the size of the risk area during coronary occlusion and the size of the region of persistent contrast enhancement after MBESEL injection (y=0.98x-0.01, r=0.92, risk area size data not shown).

F. Confirmation of Leukocyte Adhesion Molecule Upregulation

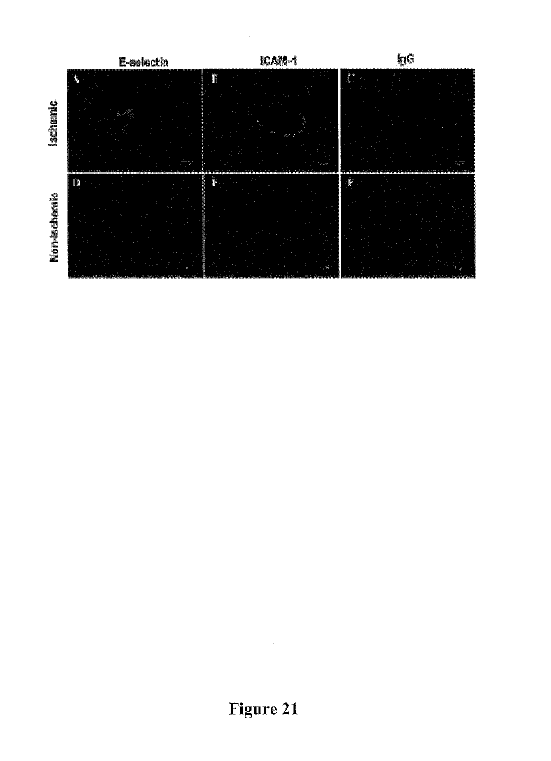

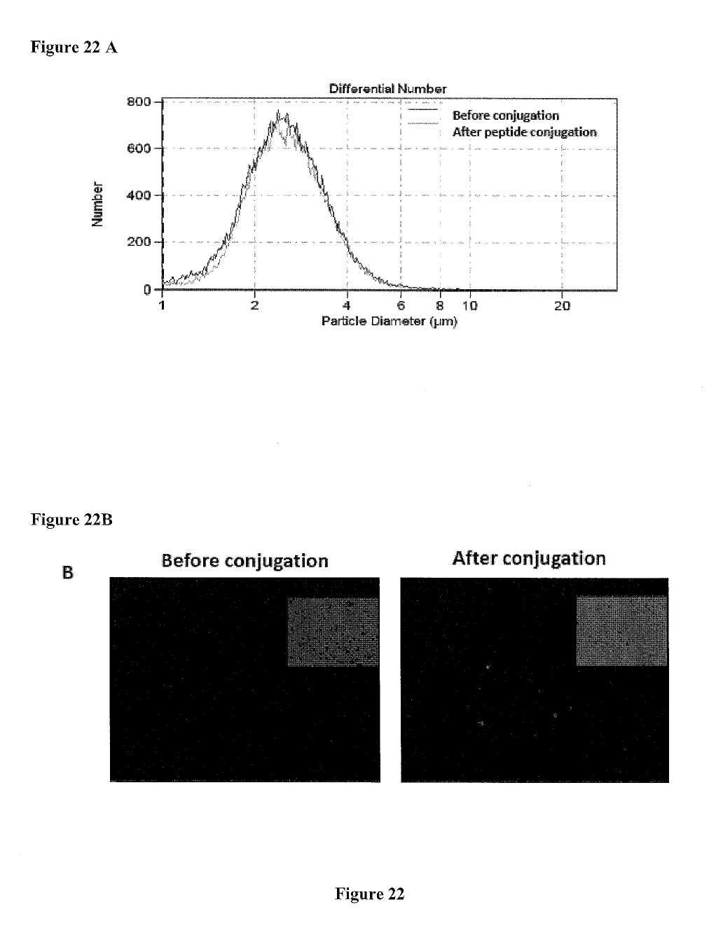

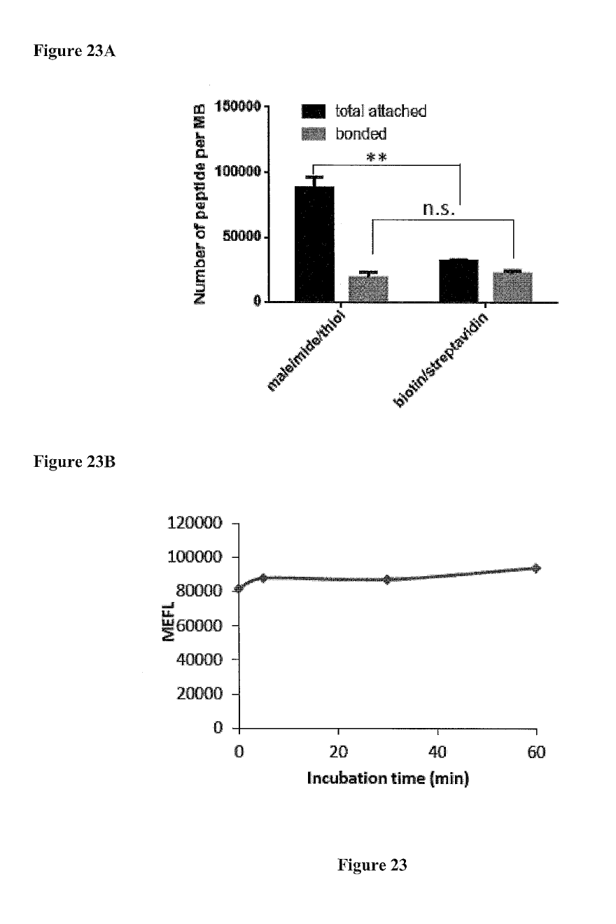

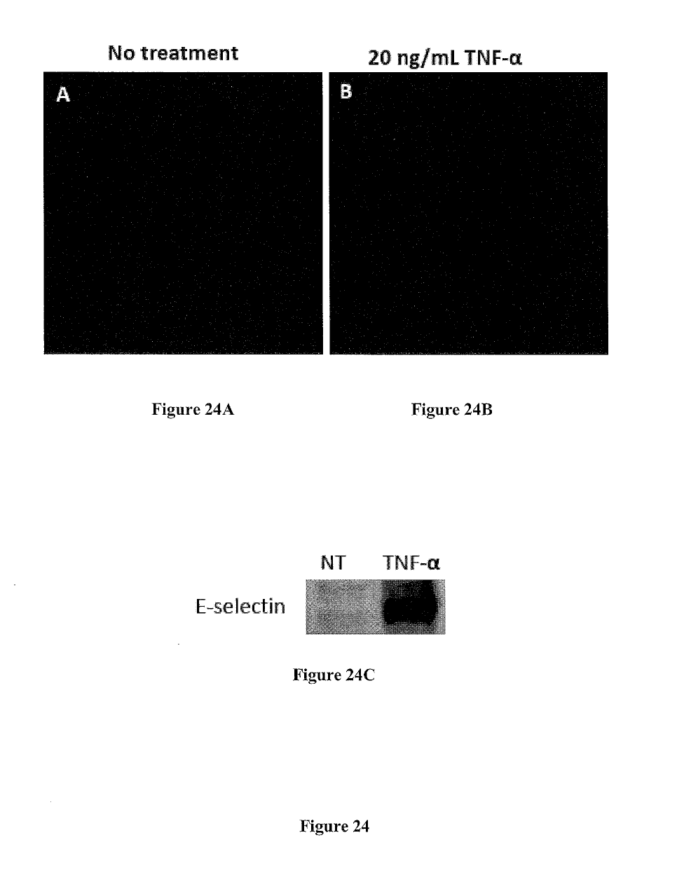

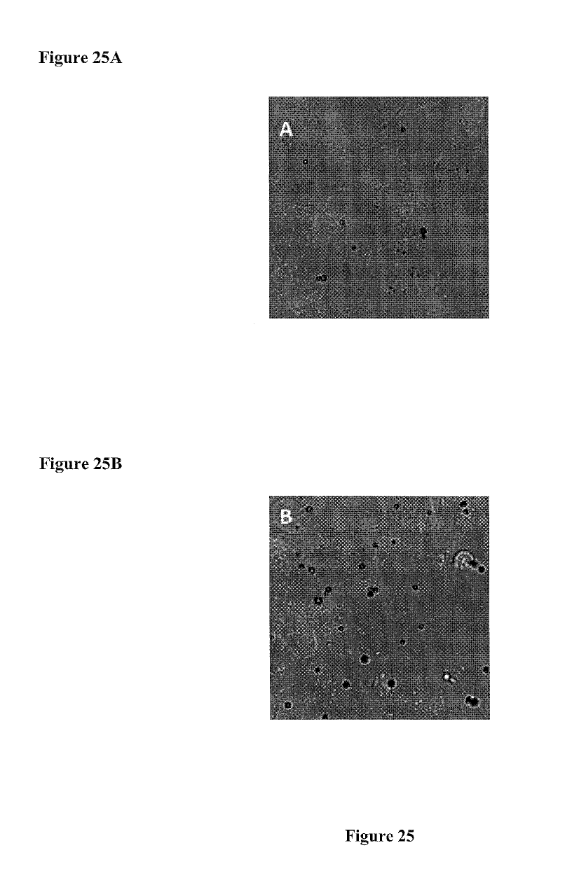

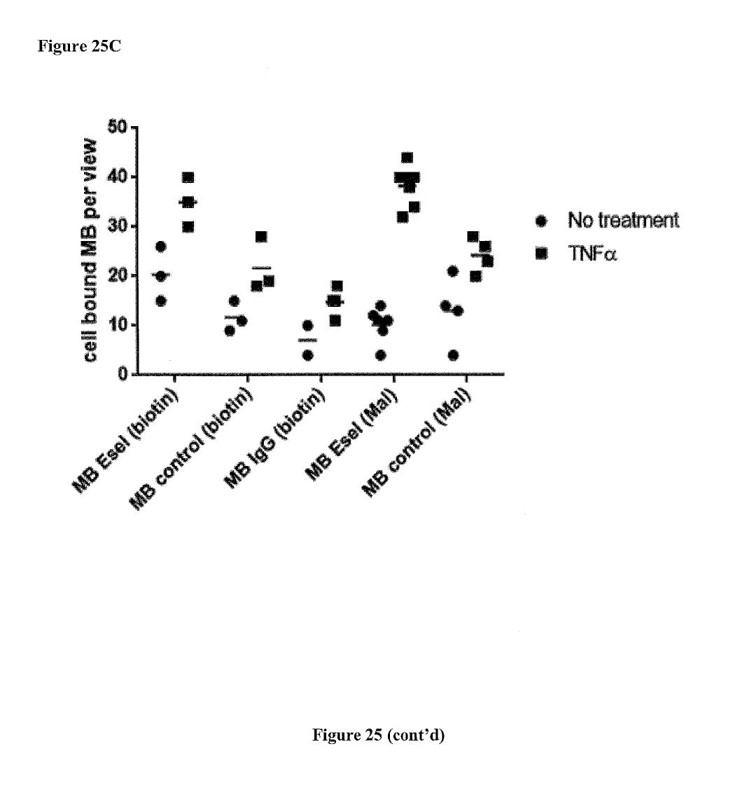

Immunofluorescent staining in all rats consistently confirmed microvascular E-selectin and ICAM-1 expression in postischemic myocardium that was minimally seen in the nonischemic bed. FIG. 21. Quantitative RT-PCR in all six rats indicated a 3.3-fold (p<0.03) and 3.2-fold (p<0.02) upregulation of E-selectin and ICAM-1 mRNA, respectively, in the ischemic bed relative to the nonischemic bed of the same heart.

IV. Clinical Relevance For Targeted Microbubbles

The main finding of this study is that postischemic myocardium can be identified with ultrasound imaging using E-selectin-targeted MBs. Importantly, the MBs were targeted via a short peptide having specific affinity to human E-Selectin, which has distinct clinical advantages over previously used antibody^{16,17} or carbohydrate-targeting strategies,^{3,18} which confer immunogenicity or require complex synthetic chemistry, respectively. Thus, these data set the stage for human trials of bedside ischemic memory imaging in patients presenting with chest pain of possible ischemic etiology.

A. Challenges in Diagnosing ACS

Chest pain of possible cardiac origin accounts for 5 to 6 million emergency department visits annually.¹⁹ Due to the resolution of symptoms by the time of presentation, atypical symptoms, and/or a nondiagnostic electrocardiogram, the diagnosis of true ACS can be elusive. Patients with true myocardial ischemic who are inappropriately discharged from the emergency department suffer high mortality and morbidity.^{20,21} To avoid missing the diagnosis of ACS, physicians conservatively admit two to three patients for every one with true ACS, resulting in a large economic burden. Despite a conservative admission strategy, the diagnosis of true ACS is still missed in 2 to 7% of patients, underscoring the limitations in current tools for accurately identifying ACS. A rapid and noninvasive method for detecting true ongoing or recent myocardial ischemia would have both a high economic and a clinical impact. However, current methods such as electrocardiography,²² troponin detection,²³ stress radionuclide single-photon emission computed tomography (SPECT),²⁴ and cardiac computed tomographic (CT) angiography²⁵ have limitations in diagnosing ACS. In particular, the most commonly employed imaging method for triaging patients presenting with chest pain, stress perfusion testing, detects impairment in flow reserve, which occurs in, and therefore does not distinguish among, stable coronary stenotic lesions,²⁶ recent myocardial ischemia from an unstable plaque,²⁷ or previous infarction.²⁸

In one embodiment, the present invention contemplates a method for the clinical diagnosis of acute coronary syndrome (ACS) in patients presenting with chest pain of possible ischemic origin using ultrasound molecular imaging of an E-selectin targeted ultrasound contrast agent (e.g., an E-selectin conjugated microbubble).

The accurate and rapid diagnosis of ischemic chest pain in the emergency department (ED) is a clinical and economic challenge. Chest pain of possible cardiac origin accounts for 5-6 million ED visits annually, with an annual cost estimated in the US of several billion dollars. Due to the resolution of symptoms by the time of presentation to the ED, atypical symptoms, and/or a non-diagnostic ECG, the accurate diagnosis ACS can be elusive. Cardiac biomarkers in patients with acute myocardial infarction are often normal initially, and those with unstable angina may not have a rise in serum biomarkers at all. Patients with true ACS who are inappropriately discharged from the ED suffer high mortality and morbidity. Hence, physicians defensively admit to the hospital 2 to 3 chest pain patients for every one with true ACS, resulting in a large economic burden ($1.7 million per life saved). Yet despite this conservative hospital admission strategy, the diagnosis of ACS is still missed in 2-6% of patients, underscoring the limitations in current tools for accurately identifying ACS. A rapid non-invasive method for detecting recent or ongoing myocardial ischemia would have both a high economic as well as clinical impact. However, current methods for detecting ACS, such as ECG, troponin detection, stress radionuclide single photon emission tomography, or cardiac CT angiography, have significant limitations, creating both a compelling need, as well as opportunity, for innovative solutions. To address these challenges, the present invention contemplates a rapid bedside ultrasound molecular imaging method to detect ACS.

In one embodiment, an ultrasound contrast agent (e.g., for example, a population of microbubbles approximately 2-3 microns in diameter) targeted to bind specifically to the leukocyte adhesion molecule (i.e., for example, E-selectin) which may be upregulated in acutely ischemic microvascular endothelial cells. In one embodiment, the present invention contemplates a formulation comprising an E-selectin targeted imaging probe as a double layer microbubble comprised of an outer shell of crosslinked human albumin, an inner layer of biodegradable polymer (poly-DL-lactide, PDLLA), and encapsulated nitrogen gas. Although it is not necessary to understand the mechanism of an invention it is believed that a peptide AF10166 (H₂N-DITWDQLWDLMK-COOH), previously identified by phage display to have specific affinity to human E-selectin, is used as the cellular targeting moiety. Consequently, after intravenous injection, the E-selectin conjugated microbubble binds to recently ischemic myocardial microcirculation, resulting in a persistent ultrasound signal during bedside echocardiography. Non-ischemic myocardium (e.g. patients whose chest pain is not ischemic in origin) will not take up the targeted microbubbles, resulting in an echocardiographic image that does not show persistent ultrasound signal.

The data presented herein demonstrates in vitro and in vivo studies establishing that E-selectin (Esel) targeted microbubbles (MB) bind to activated endothelial cells overexpressing E-selectin, including, but not limited to:
(A) In in vitro flow chamber studies specific binding of the targeted microbubbles to cultured endothelial cells overexpressing E-selectin is demonstrated;
(B) E-selectin targeted microbubble adhesion to inflamed endothelium in vivo was confirmed by direct visualization of microbubble interactions with inflamed rat cremaster muscle microcirculation; and
(C) Induced rat myocardial ischemia followed by reperfusion and ultrasound molecular imaging several hours after reperfusion, mimics a human patient who has prolonged (e.g., several hours) but transient chest pain.

Echocardiographic images confirmed not only the occurrence of recent ischemia after intravenous injection of E-selectin targeted microbubbles but also mapped the spatial extent of ischemia (i.e., the area at risk was clearly delineated by the appearance of persistent contrast enhancement in the risk area during echocardiography). Leng et al., “Ultrasound detection of myocardial ischemic memory using an E-selectin targeting peptide amenable to human application” J Molecular Imaging 13:1-9 (2014). Proof of concept in vivo studies for ischemic memory imaging using the sLEx targeting ligand (sLex) used targeted microbubble binding to leukocyte adhesion molecules. Villanueva et al., “Myocardial ischemic memory imaging using molecular echocardiography” Circulation 115:345-352 (2007).

B. Molecular Imaging

A molecular imaging approach to detect acute myocardial ischemia has inherent advantages over anatomic (cardiac CT) or physiologic (stress testing) imaging methods. An imaging probe targeted to a molecular or metabolic consequence of ischemia potentially offers higher specificity in detecting ACS as a cause of chest pain. For example, a radioactive fatty acid tracer, b-methyl-p-[¹²³I]-iodophenyl-pentadecanoic acid (BMIPP) was used to detect ischemia-induced shift to glucose metabolism as a marker of ischemic memory.²⁹

In patients with thallium²⁰¹ treadmill exercise-induced ischemia, BMIPP injection and SPECT up to 22 hours after the ischemia showed reduced BMIPP uptake in myocardial segments with reduced ²⁰¹thallium uptake. Although promising, this method entails patient exposure to radioactivity and specialized cameras and is still in investigational stages.

In some embodiments described herein, MBs are used as an acoustic imaging probe to bind endothelial leukocyte adhesion molecules that are upregulated after myocardial ischemia and reperfusion. E-selectin was chosen as a preferred adhesion molecule because the peptide mediates an initial weak attachment of activated leukocytes to ECs and neutrophil recruitment to the myocardium.³⁰ E-selectin requires de novo synthesis and is transcriptionally regulated, appearing on endothelium within hours of ischemia and reperfusion, reaching a maximum at 2 to 4 hours after stimulation, and decreasing by 24 hours thereafter.^{31,32} Cell surface expression of E-selectin protein parallels that of the mRNA. The data shown herein demonstrates immunofluorescent staining and RT-PCR showing E-selectin upregulation only in postischemic myocardium, making E-selectin a strong candidate for molecular imaging of ischemic memory. Furthermore, the time course of E-selectin expression—lasting hours after ischemic insult—offers a clinically practical time window for useful imaging, potentially allowing for diagnostic molecular imaging of ischemic memory for the significant number of patients who do not present to the emergency department immediately after the onset of chest pain.

C. Polymer MB/Peptide Construct Advantages

Ultrasound studies targeting leukocyte adhesion molecules have been previously reported. MBs bearing monoclonal antibodies targeted to P-selectin ultrasonically detected recent myocardial ischemia in mice.¹⁶ Previously targeted P-selectin has used a native ligand sLeX to identify recent ischemia.³ Unlike the present invention, however, these earlier studies used MB formulations that are not optimally suited for human translation due to the immunogenicity of antibodies or complex carbohydrate chemistry involved in the synthesis and MB conjugation of sLeX. Davidson and colleagues recently reported ischemic memory imaging using selectin-targeted MBs bearing a P-selectin glycoprotein ligand, which may have promise for human translation, but synthesis of the ligand involves complex recombinant methods and purification and is costly.³³

Some embodiments disclosed herein are the first to use a polymer MB conjugated to a peptide for molecular imaging. This construct may overcome some of the current hurdles to clinical translation of previously described ultrasound contrast agents for molecular imaging. The dual-layer MB shell results in high stability, high echogenicity, and low immunogenicity.¹⁰ The short peptide ligand offers simple chemistry, should not have the immunogenicity of monoclonal antibodies, and, unlike other ligands reported for UMI of ischemic memory, is inexpensive. AF10166 is a 12-mer peptide with high affinity (IC₅₀=4.4 nM) to human E-selectin. Binding of AF10166 to human E-selectin blocks neutrophil adhesion in both static and flow-cell assays.¹¹ When the AF10166 peptide was attached to the MB shell, the targeted MBs bound to inflamed cultured ECs under flow conditions. Postischemic myocardium was persistently echogenic after targeted MB injection, consistent with microvascular adhesion of the targeted MBs. Given that the peptide was identified using phage display against human cells, binding to human E-selectin in vivo might be even more robust, resulting in images superior to those obtained in the current rat study. It should be noted that other myocardial inflammatory conditions associated with E-selectin overexpression, such as acute myocarditis or heart transplant rejection, would also be detected by our imaging method. However, these conditions would show a spatial pattern of diffuse, persistent contrast enhancement, whereas recent acute ischemia from epicardial coronary obstruction results in contrast persistence in a vascular territory distribution¹⁴.

D. Treatment Of Inflamation

In one embodiment, the present invention contemplates a method for treating LPS-induced tissue inflammation. See, for example, Example XI. The use of targeted MB Esel(MAL) demonstrated that: a) Esel increased to 2.28±1.32% of GAPDH 4 hours post LPS injection as compared with no LPS treatment at 0.11±0.06% (n=3 for each group); b) ICAM increased to 6.09±3.14% of GAPDH 4 hours post LPS injection as compared with no LPS treatment at 0.29±0.11% (n=3 for each group); and c) injection of LPS can be used as a model of global inflammation with E-sel overexpression.

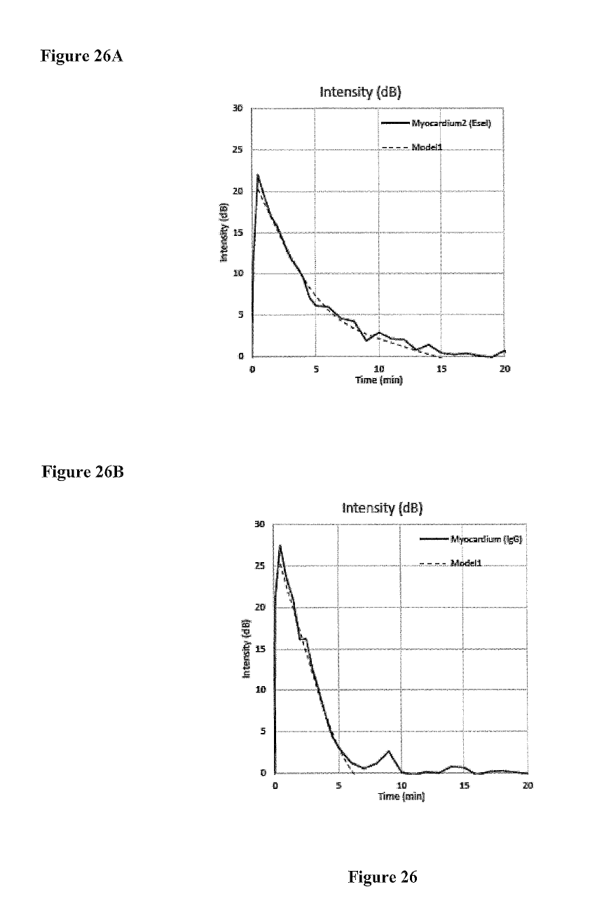

The data presented herein demonstrated that a targeted MB has higher retention in the myocardium as compared with a control IgG MB. For example, at 6 minutes post-injection, the targeted MB myocardial concentration was 185% more as compared with a control IgG MB injection. cf, FIG. 26A and FIG. 26B. The data was calculated using a two-phase washout model: Iₜᵢₛₛᵤₑ=A_{f}e^{-aft}+Aᵣe^{-art} where:

Iₜᵢₛₛᵤₑ=linearized tissue intensity;

Af, Ar=free circulating MB concentration and retained MB concentration, respectively;

af, ar=free circulating MB washout rate and retained MB washout rate, respectively;

Hemodynamic curves (e.g., linearized intensity vs time curves) fitted to the washout model above, identified that retained MB in the myocardium (e.g., Ar term) was 330% with targeted MB injection as compared with control MB injection. Although it is not necessary to understand the mechanism of an invention, it is believed that based upon conventional subtraction methods of imaging-burst-imaging a later time point might result in better detection of adhesion and a lower MB dose may provide improved retention.

V. Polymer-Based Microbubbles

In one embodiment, the microbubbles according to the present invention may have a bi-layered shell. For example, an outer layer of the shell may comprise a biologically compatible material or biomaterial. Although it is not necessary to understand the mechanism of an invention, it is believed that that biological surface material is advantageous to stability as the microbubble surface is exposed to blood and other tissues within the body. The inner layer of a microbubble shell may comprise a biodegradable polymer. For example, the biodegradable polymer comprises a synthetic polymer, which may be tailored to provide the desired mechanical and acoustic properties to the shell or provide drug delivery properties.

A. Polymer-Based Dual Shell Layer Microbubble Compositions

In some embodiments, the compositions are polymer-based microbubble ultrasound contrast agents comprising a hollow core of a gas including, but not limited to, air or nitrogen. In some embodiments, compositions are polymer-based rupturable microbubbles (e.g., by a low intensity ultrasound energy). Microbubbles are constructed herein such that the majority of those prepared in a composition will have diameters within the range of about one to ten microns in order to pass through the capillary system of the body.

Since the presently disclosed polymer-based microbubbles have an outer and inner layer, these layers can be tailored to serve different functions. For example, an outer shell which is exposed to the blood and tissues may serve as a biological interface between the microbubbles and the body. Thus, an outer shell comprises a biocompatible material which is typically amphiphilic, that is, has both hydrophobic and hydrophilic characteristics. In other embodiments, blood compatible materials are particularly preferred including, but not limited to, collagen, gelatin or serum albumins or globulins, proteins, glycosoaminoglycans such as hyaluronic acid, heparin and chondroitin sulphate and combinations or derivatives thereof.

In other embodiments, the outer shell layer may comprise a synthetic biodegradable polymer including, but not limited to, polyethylene glycol, polyethylene oxide, polypropylene glycol and combinations or derivatives. As the outer polymer shell layer is typically amphiphilic, as well as having a chemistry which allows charge and chemical modification, this surface versatility allows for modifications including, but not limited to, altering outer shell electrical charge. Electrical charge alterations may be provides by selecting a type A gelatin having an isoelectric point above physiological pH, or by using a type B gelatin having an isoelectric point below physiological pH.

Modifications to polymer-based outer shell layer surfaces may also be chemically modified to enhance biocompatibility, such as by PEGylation, succinylation or amidation, as well as being chemically binding to the surface targeting moiety for binding to selected tissues. The targeting moieties may be antibodies, cell receptors, lectins, selecting, integrins or chemical structures or analogues of the receptor targets of such materials. The mechanical properties of the outer layer may also be modified, such as by cross linking, to make the microbubbles suitable for passage to the left ventricle, to provide a particular resonant frequency for a selected harmonic of the diagnostic imaging system, or to provide stability to a threshold diagnostic imaging level of the ultrasound radiation.

In some embodiments, the polymer-based microbubble comprises an inner shell comprising a biodegradable polymer. In one embodiment, the biodegradable polymer is a synthetic polymer. Although it is not necessary to understand the mechanism of an invention, it is believed that an inner shell provides improved mechanical stabilization properties to the microbubble which are not provided or insufficiently provided by an outer layer alone, or enhances mechanical properties not sufficiently provided by an outer layer alone, without being constrained by surface property requirements. For example, a biocompatible outer layer of a cross-linked proteinaceous hydrogel can be physically supported using a high modulus synthetic polymer as an inner layer. In one embodiment, a high modulus synthetic polymer may be selected for its modulus of elasticity and elongation, which define the desired mechanical properties.

In some embodiments, a biodegradable polymers includes, but is not limited to, polycaprolactone, polylactic acid, polylactic-polyglycolic acid co-polymers, co-polymers of lactides and lactones, such as epsilon-caprolactone, delta-valerolactone, polyalkylcyanoacrylates, polyamides, polyhydroxybutryrates, polydioxanones, poly-beta-aminoketones, polyanhydrides, poly-(ortho)esters, polyamino acids, such as polyglutamic and polyaspartic acids or esters of polyglutamic and polyaspartic acids. Langer, et. al. (1983) Macromol. Chem. Phys. C23, 61-125.

Although it is not necessary to understand the mechanism of an invention, it is believed that a polymer-based microbubble inner layer permits mechanical property modification that cannot be provided by an outer layer alone. In embodiments comprising a polymer-based microbubble ultrasonic contrast agent, an inner layer comprises a thickness which is no larger than is necessary to meet a minimum mechanical constraint. Although it is not necessary to understand the mechanism of an invention it is believed that a combined thickness of the outer and inner layers of the polymer-based microbubble shell depend, in part, on a pre-determined mechanical stability property. Nonetheless, it is believed that a total shell thickness may range between approximately 25 nm to 750 nm.

B. Polymer-Based Dual Shell Layer Microbubble Synthesis

In some embodiments, polymer-based microbubbles may be prepared by an emulsification process to control the sequential interfacial deposition of shell materials. Due to the amphiphilicity of a material forming an outer layer, stable oil/water emulsions may be prepared having an inner phase to outer phase ratio approaching 3:1, without phase inversion, which can be dispersable in water to form stable organic phase droplets without the need for surfactants, viscosity enhancers or high shear rates.

Two solutions may be prepared: i) an aqueous solution of the outer biomaterial; and ii) a solution comprising an inner layer polymer, an inner layer polymer solvent comprising a relatively volatile water-immiscible liquid, and an inner layer polymer non-solvent comprising a relatively non-volatile water-immiscible liquid. In one embodiment, the inner layer polymer solvent comprises a C5-C7 ester compound (e.g., for example, isopropyl acetate). In one embodiment, the inner layer polymer non-solvent comprises a C6-C20 hydrocarbon (e.g., for example, decane, undecane, cyclohexane and/or cyclooctane).

The two above solutions are combined and agitated so that the inner layer polymer fully dissolves and the two solvents become miscible. The polymer solution (organic phase) is slowly added to the biomaterial solution (aqueous phase) to form a liquid foam. In one embodiment, about three parts of an organic polymer solution having a concentration of about 0.5 to 10 percent of a polymer is added to one part of an aqueous biomaterial solution having a concentration of about 1 to 20 percent of a biomaterial. The relative concentrations of the solutions and the ratio of organic phase to aqueous phase utilized in this step essentially determine the size of the final microbubble and wall thickness. After thorough mixing, the liquid foam is dispersed into water and typically warmed to about 30-35° C. with mild agitation. While not intending to be bound by a particular theory, it is believed that a biomaterial in the foam disperses into the warm water to stabilize an emulsion of the polymer in the organic phase encapsulated within a biomaterial envelope. To render the biomaterial envelope water insoluble, a cross linking agent, such as glutaraldehyde, may be added to the mixture to react with the biomaterial envelope and render it water insoluble, thereby stabilizing the outer shell. Other cross-linking agents may be used including, but not limited to, carbodiimide cross-linkers.

In one embodiment, an inner core is formed comprising a polymer solution, a solvent and a non-solvent. Because the solvent and non-solvent have different volatilities, the more volatile solvent evaporates, or is diluted, and the polymer precipitates in the presence of the less volatile non-solvent. This process forms a film of precipitate at an interface with an inner surface of a biomaterial shell, thus forming an inner shell of a microbubble after the more volatile solvent has been reduced in concentration either by dilution, evaporation or the like. The core of the microbubble then contains predominately an organic non-solvent. The microbubbles may then be isolated by centrifugation, washed, formulated in a buffer system, if desired, and dried. Typically, drying by lyophilization removes not only the non-solvent liquid core but also the residual water to yield gas-filled hollow microbubbles.

In some embodiment, a microbubble surface is modified. In one embodiment, the modification passivates the microbubble surface against macrophages or the reticuloendothelial system (RES) in the liver. This may be accomplished, for example, by chemically modifying the surface of the microbubble to be negatively charged since negatively charged particles appear to better evade recognition by macrophages and the RES than positively charged particles. Also, the hydrophilicity of the surface may be changed by attaching hydrophilic conjugates, such as polyethylene glycol (PEGylation) or succinic acid (succinylation) to the surface, either alone or in conjunction with the charge modification.

A biomaterial microbubble surface may also be modified to provide targeting agents. The surface targeting agents may comprise, for example, antibodies or biological receptors. For example, if a microbubble were modified with targeting agents directed to tumors and were hollow, microbubbles could be used for ultrasound detection to enhance diagnosis of a tumor.

The microbubbles may also be sized or processed after manufacture. This is an advantage over lipid-like microbubbles which may not be subjected to mechanical processing after they are formed due to their fragility. After preparation, but prior to use, the microbubbles may take the form of a lyophilized cake. A later reconstitution of the microbubbles may be facilitated by lyophilization with bulking agents which provide a cake having a high porosity and surface area. The bulking agents may also increase the drying rate during lyophilization by providing channels for the water and solvent vapor to be removed. This also provides a higher surface area which would assist in the later reconstitution. Typical bulking agents are sugars such as dextrose, mannitol, sorbitol and sucrose, and polymers such as PEG's and PVP's.

It is undesirable for microbubbles to aggregate, either during formulation or during later reconstitution of the lyophilized material. Aggregation may be minimized by maintaining a pH of at least one to two pH units above or below the isoelectric point (Pᵢ) of the biomaterial forming the outer surface. A charge on the microbubble surface is determined by the pH of the formulation medium. Thus, for example, if the surface of a biomaterial has a Pᵢ of 7 and the pH of the formulation medium is below 7, the microbubble will possess a net positive surface charge. Alternatively, if the pH of the formulation medium is greater than 7, the microbubble would possess a negative charge. A maximum potential for aggregation exists when a pH of the formulation medium approaches the Pᵢ of a biomaterial used in an outer shell. Therefore, a formulation medium pH at least one to two units above or below the Pᵢ of a microbubble surface minimizes microbubble aggregation. As an alternative, a microbubble may be formulated at or near the Pᵢ with surfactants to stabilize against aggregation.

In one embodiment, an injectable microbubble population formulation comprises a physiologically compatible buffer. Bulking agents may be utilized during microbubble lyophilization to control the osmolality of the final formulation for injection. An osmolality, other than physiological osmolality, may be desirable during lyophilization to minimize aggregation. However, when formulating the microbubbles for use, the volume of liquid used to reconstitute the microbubbles must take this into account.

Other additives may be included in order to prevent aggregation or to facilitate dispersion of the microbubbles upon formulation. Surfactants may be used in the formulation such as poloxomers including, but not limited to, polyethylene, glycol-polypropylene, glycol-polyethylene, and/or glycol block co-polymers. Water soluble polymers also may assist in the dispersion of the microbubbles, such as medium molecular weight polyethylene glycols and low to medium molecular weight polyvinylpyrolidones.

It will be realized that various modifications of the above-described processes may be provided without departing from the spirit and scope of the invention. For example, the wall thickness of both an outer and inner layer may be adjusted by varying concentrations of the components in the microbubble-forming solutions. The mechanical properties of the microbubbles may be controlled, not only by the total wall thickness and thicknesses of the respective layers, but also by selection of materials used in each of the layers by their modulus of elasticity and elongation, and degree of cross-linking of the layers. Upon certain conditions involving rapid deposition of an inner polymer, a very low inner polymer content porosity of the inner polymer shell is observed. The pores range from approximately 0.1 to 2 micron in diameter as observed under electron microscopy.

Mechanical properties of the layers may also be modified with plasticizers or other additives. Adjustment of the strength of the shell may be modified, for example, by the internal pressure within the microbubbles. In particular, by appropriately adjusting the mechanical properties, the microbubbles may be made to remain stable to threshold diagnostic imaging power, while being rupturable by an increase in power and/or by being exposed to its resonant frequency. The resonant frequency can be made to be within the range of transmitted frequencies of diagnostic body imaging systems or can be a harmonic of such frequencies. During the formulation process the microbubbles may be prepared to contain various gases, including blood soluble or blood insoluble gases. It is a feature of the invention that microbubble compositions may be made having a resonant frequency greater or equal to 2 MHz, and typically greater or equal to 5 MHz.

### Experimental

### EXAMPLE I

### Conjugation of Targeting Proteins to a Polymer Shell Microbubble

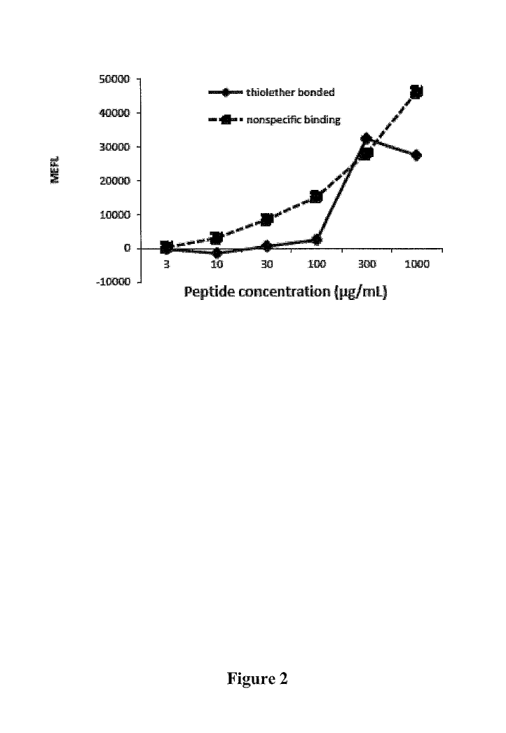

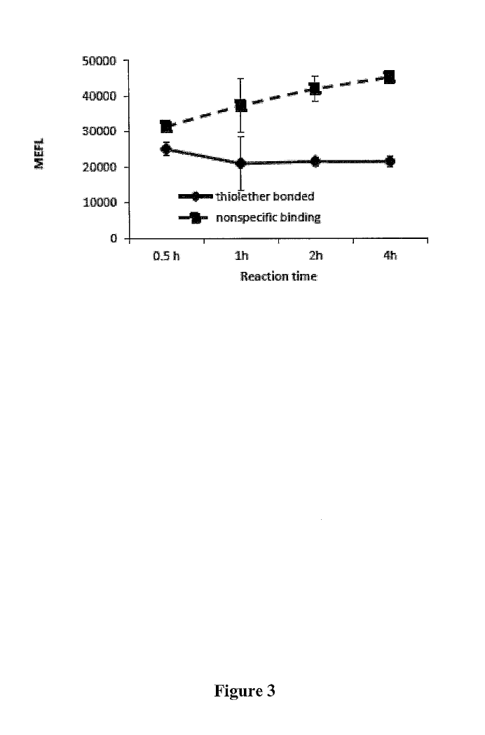

Using the maleimide conjugation process described herein (FIG. 1), a significant amount of peptides was observed to be non-specifically bound to MB, which could result in a low-yield conjugation process. Therefore, the conjugation condition was optimized by using fluorophore labeled peptide (DITWDQLWDLMK-FITC; SEQ ID NO:3) as a surrogate. The number of peptides bound to each microbubble was measured with flow cytometry using beads with known number of fluorophores as standard. FIGS. 2 and 3.

### EXAMPLE II

### Detection of Recent Myocardial Ischemia

### Preparation of a Targeted Microbubble

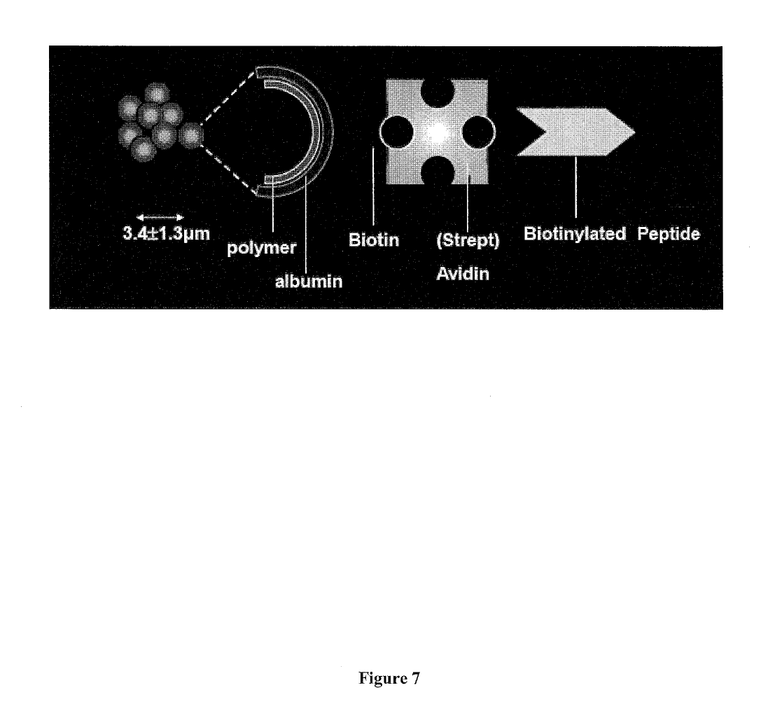

Targeted biodegradable polymer MB (3.4±1.3μm) were prepared bearing a 12-mer synthetic peptide on the surface with specific E-selectin affinity (MBESEL). See, FIG. 7. Control MB had scrambled peptide (MBCTL) or non-specific IgG (MBIgG). MB adhesion to cultured rat endothelial cells (EC) was assessed in a parallel plate flow system (n=3/condition). Intravital microscopy of rat cremaster microcirculation was performed after i.v. injection of each MB type under basal or activated conditions (scrotal TNF 5μg) (n=3 rats/condition). To determine if binding events could be imaged, 6 rats had 15 min coronary artery occlusion. After 4 hrs reflow, each rat received separate i.v. boluses of MBESEL, MBCTL, and MBIgG. Non-linear echo imaging was performed 3 and 3.5 min later. Videointensity (VI) differences between the 2 time points was attributed to target-specific signal.

### Results

MBESEL adhesion was higher to activated vs normal EC (17±2 vs 5±3 MB/EC, p<0.01) in vitro. There was no difference in adhesion to activated vs normal EC when EC were perfused with MBCTL (5±2 vs 4±1 MB/EC, p=0.41) or MBIgG (2±1 vs 2±1/EC, p=0.48). Intravital microscopy showed greater adhesion of MBESEL to inflamed vs non-inflamed microcirculation (96±23 vs 34±8 MB/field, p<0.03), and minimal adhesion of MBCTL or MBIgG under any condition. Myocardial VI in the post-ischemic bed after MBESEL was higher vs the nonischemic bed (12±3 dB vs 4±1 dB, p<0.02), and higher than that after MBCTL (4±1 dB, p<0.03) or MBIgG (2±0 dB, p<0.02).

### EXAMPLE III

### Shear Stress Platform for Testing Targeted Microbubble Stability

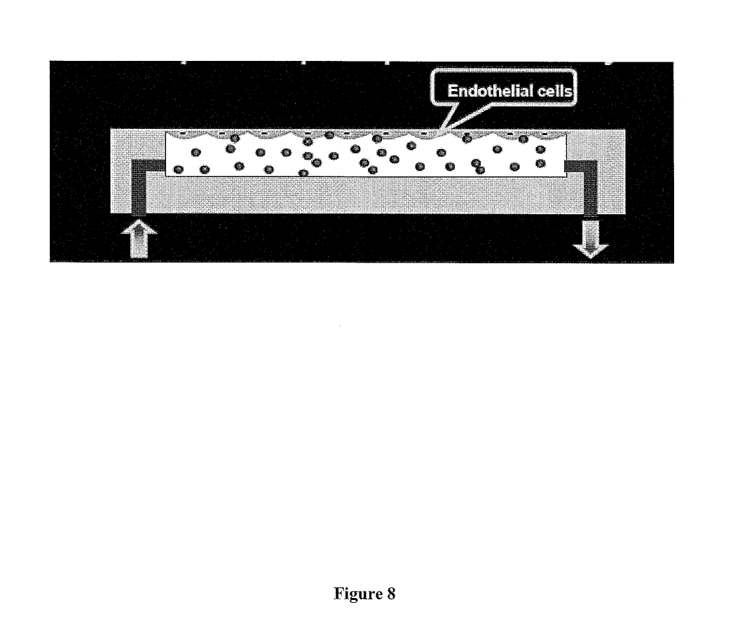

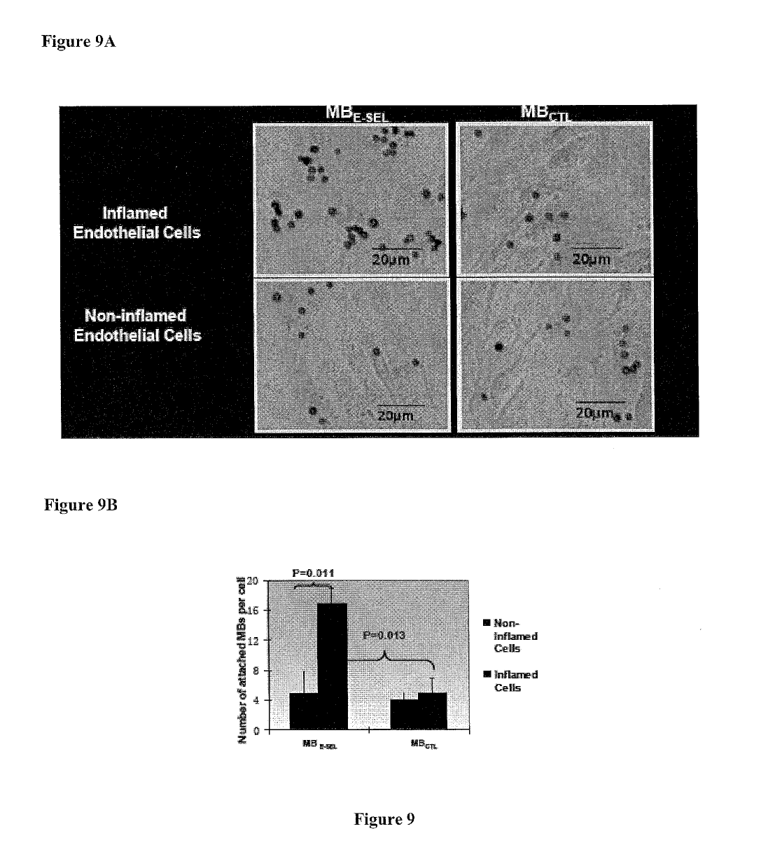

Cultured rat heart microvessel endothelial cells were placed on glass cover slips. The cells were then stimulated with IL-1β (2 ng/ml) for 4 hrs and then perfused with 1 mL microbubbles (5×10⁶ /mL)-MBE-SEL or MBCTL. These microbubbles were then subjected to a wall shear rate of 200 sec-1. See, FIG. 8. 20 random fields (n=3) were assessed. The data demonstrates the superior binding of the E-selection targeted microbubbles in inflamed and non-inflamed cells. FIGS. 9A and 9B.

A parallel plate perfusion chamber was used to quantify MB adhesion to cultured endothelial cells (ECs).12 Rat heart microvascular ECs (VEC Technologies, Rensselaer, N.Y.) were grown to confluence on 25 mm 3 75 mm glass coverslips. Cells were incubated for 5 hours with 5 ng/mL interleukin-1b (Sigma-Aldrich, St. Louis, Mo.) to cause activation. Coverslips with normal or IL-1b-activated ECs (n =5; 3/condition) were mounted in the chamber and perfused with one of the three MB species (5×10⁶ MB in 1 mL culture medium) at a wall shear rate of 200 s-1, followed by an additional 3 mL of plain culture medium. The chamber was then mounted on a microscope (Nikon TE200) and 20 random bright-field images (3 100) of the ECs were captured (ORCA285, Hamamatsu, Bridgewater, N.J.). The mean number of attached MBs/ECs was counted offline.

### EXAMPLE IV

### Intravital Microscopy Study

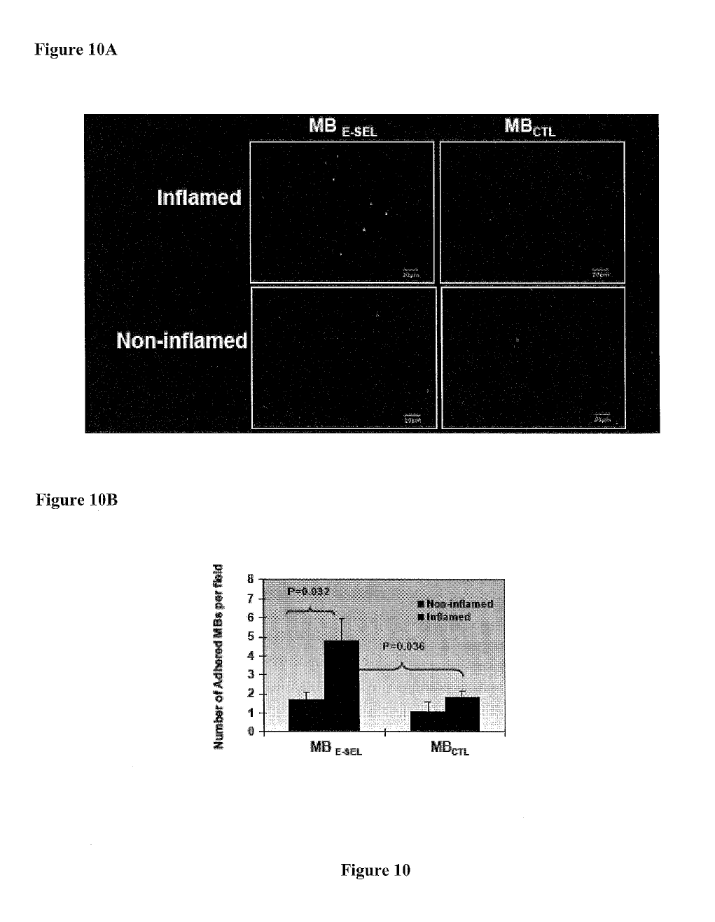

An intravital microscopy of rat cremaster muscle microcirculation was performed after intravenous injection of 1×10⁸ MBE-SEL or MBCTL (n=3 per condition). Non-inflamed: 4 hrs after intrascrotal injection of saline. Inflamed : 4 hrs after intrascrotal injection of TNF-α (5 μg). Real-time images were captured and microbubble adhesion was quantified in venules at 5 minutes after microbubble injection in 20 random fields. See, FIGS. 10A and 10B.

Microscopy of the rat cremaster muscle microcirculation was used for real-time visualization of MB adhesion in vivo. Sprague Dawley rats (100-120 g) received intrascrotal injection of either tumor necrosis factor a (n=9, 5 μg, Thermo Scientific, Waltham, Mass.) or 1 mL saline (n=9). Four hours later, the cremaster muscle was exteriorized and mounted on an inverted fluorescent microscope (Nikon TE200). Each rat received one intravenous injection (1×10⁸ MB) of either MBESEL or control MBs (MBCTL or MBIgG) in 200 mL saline, followed by a 200 mL saline flush (n=5, 3 rats per MB type/inflammatory status combination, total 6 groups). Five minutes after injection, bright-field microscopic imaging of 20 random fields was performed to identify microvessels, and epi-illumination fluorescent imaging was performed to identify MBs, followed by pentobarbital overdose. Images were analyzed offline for MB adhesion per field of view, defined as MB immobility lasting≧30 seconds.

### EXAMPLE V

### Rat Heart Ischemia-Reperfusion Study

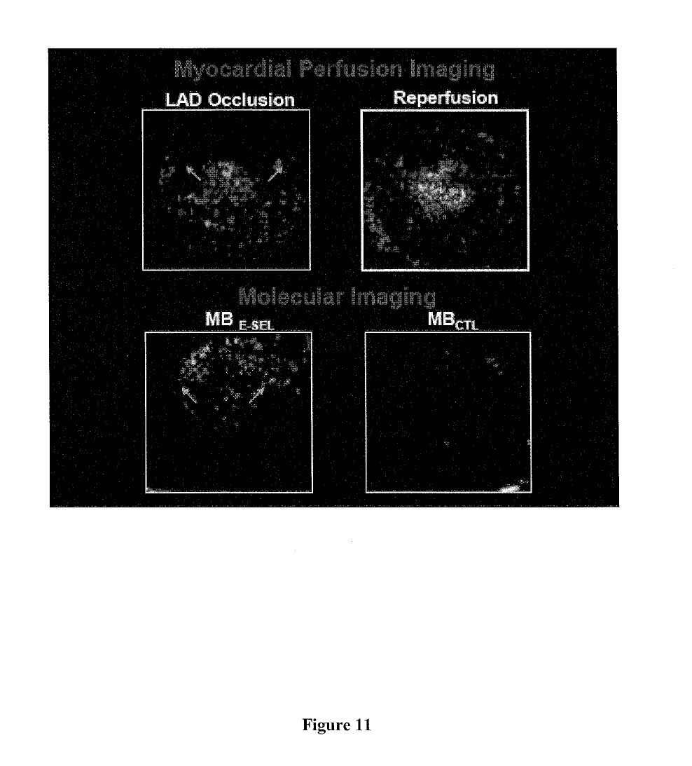

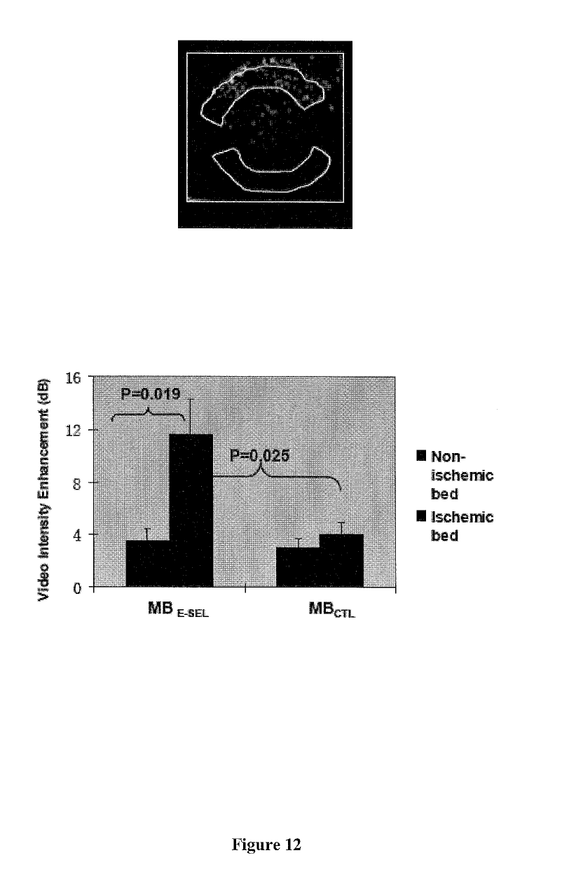

An open chest rat model of myocardial ischemia-reperfusion (n=6) was performed where the left anterior descending coronary artery was occluded for 15 minutes then reperfused. See, FIG. 11. At 4 hrs of reflow, the rat received separate i.v. injections of MBE-SEL or MBCTL (2.5×10⁷ MBs). Triggered ultrasound imaging (8 MHz, MI 0.6, Sequoia CPS, Siemens) was performed 3 min after injection, followed by 5 destruction frames (MI 1.9) and a second imaging frame (MI 0.6) at 3.5 minutes. The videointensity difference between the two imaging frames was attributed to microbubble adhesion. See, FIG. 12.

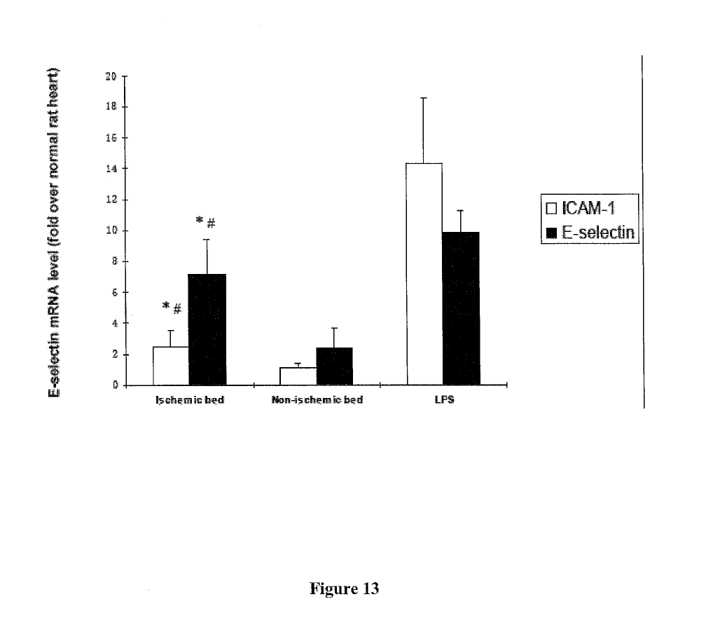

ICAM-1 and E-selectin expression during ischemia-reperfusion obtained by quantitative real-time polymerase chain reaction. See, FIG. 13. LPS was administered intravenously and heart samples were removed 5 hours post injection. All mRNA levels are expressed as fold over normal heart tissue+/−standard deviation (n=6 for each group). *_p<0.05 vs non-ischemic bed for ICAM-1. #_p<0.05 vs nonischemic bed for E-selectin.

### EXAMPLE VI

### Ultrasound Imaging Parameters

Sequoia 512 ultrasound scanner (Siemens Medical)

15L8 linear array transducer

Contrast pulse sequence imaging (CPS) at 8 MHz (bubble-specific modality)

Mechanical index for imaging=0.6

Mechanical index for microbubble destruction=1.9

Ultrasound gel used as acoustic interface (open chest)

### EXAMPLE VII

### In Vivo Studies

All protocols were approved by the Institutional Animal Care and Use Committee at the University of Pittsburgh. Wistar rats (Sprague Dawley, Harlan Laboratories, Indianapolis, Ind.) were anesthetized with intraperitoneal sodium pentobarbital (50 mg/kg) and maintained with 1.5 to 2.0% inhaled isoflurane. Body temperature was maintained at 37° C. with a heating pad. The internal jugular vein was cannulated with a 20-gauge catheter for MB injection.

### EXAMPLE VIII

### Myocardium UMI

A previously described rat model of acute myocardial ischemia and reperfusion was used for UMI of E-selectin overexpression as a marker of ischemic memory.³ Anesthetized rats (n=6, 150-200 g) were intubated and mechanically ventilated. A lateral thoracotomy was performed, and the left anterior descending (LAD) coronary artery was occluded for 15 minutes and then reperfused. After 4 hours of reflow, each rat received separate intravenous injections of MBESEL, MBCTL, and MBIgG in random order (2.5×10⁷ MBs in 200 mL saline followed by a 200 mL saline flush), and ultrasound imaging was performed as described below.

Thereafter, high mechanical index (MI) burst pulses were sent to ensure that all adhered and freely circulating MBs were destroyed prior to any subsequent injection. Typically, subsequent injections were given 5 minutes after MBs could no longer be detected in the left ventricular cavity on ultrasound imaging. The MB dosage was empirically chosen based on pilot studies assessing myocardial enhancement from various doses. At the end of the experiment, the LAD coronary artery was reoccluded, nontargeted myocardial contrast echocardiography (MCE) using plain lipid MBs was performed to delineate the risk area, and the rat was euthanized using pentobarbital overdose. Open-chest MCE in the short-axis view was performed using a contrast-specific imaging mode combining phase and amplitude modulation of the transmit signal (Cadence Contrast Pulse Sequencing, Sequoia 512, Siemens Ultrasound, Mountain View, Calif.) from a linear array transducer (15 L8) operating at 8 MHz. Probe position, dynamic range, gain settings, and focus were initially optimized and maintained constant. Ultrasound detection of bound MBs was performed using a previously described approach.^{12,13} After 3 minutes to allow MB retention and washout of unbound MBs, end-systolic imaging was performed at an MI of 0.6, followed by an ultrasound burst pulse at MI of 1.9 to destroy the MBs and then end-systolic imaging 30 seconds later at MI 0.6. The imaging time at 3 minutes post-MB injection was determined from pilot contrast imaging studies demonstrating that by 3 minutes after injection, background signal from freely circulating MBs is minimal. The post-destruction frames were subtracted from the pre-destruction frames to form a single color-coded image to represent the signal attributable to MB adhesion as previously described.¹⁴ Mean videointensity (VI) was measured in the ischemic and nonischemic beds. Using a two-way random effects model, interobserver agreement in VI measurement by three independent examiners was high (intraclass correlation coefficient of 0.99 and 0.97 for ischemic and nonischemic beds, respectively).

### EXAMPLE IX

### Quantification of Myocardial Inflammation and Infarction

Postmortem, the heart was excised and sectioned into three short-axis slices for 2,3,5-triphenyl tetrazolium chloride (TTC) staining to delineate infarction.¹⁵ Other myocardial pieces were snap-frozen in liquid nitrogen for immunohistochemistry and real-time polymerase chain reaction (RT-PCR) to quantify E-selectin expression. Immunohistochemical staining of myocardial sections was performed to detect E-selectin and ICAM-1.

Cryostat sections (5 mm) were fixed in ice-cold acetone and air dried. After blocking nonspecific protein binding with 2% bovine serum albumin in phosphate-buffered saline, sections were incubated overnight with goat polyclonal anti-rat E-selectin antibody (15 mg/mL, R&D Systems, Minneapolis, Minn.) or goat polyclonal anti-rat ICAM-1 antibody at 4° C., followed by incubation with Cy3-conjugated donkey anti-goat secondary antibody (diluted 1:2,000) or fluorescein isothiocyanate (FITC)-conjugated donkey anti-goat secondary antibody (Santa Cruz Biotechnology, Dallas, Tex.) for 2 hours at room temperature.

For negative controls, specific antibodies were replaced by nonspecific rat IgG of the same isotype. Sections were counterstained with 49,6-diamidino-2-phenylindole (Sigma-Aldrich), mounted, and examined microscopically. RT-PCR was performed to quantify myocardial messenger ribonucleic acid (mRNA) expression of E-selectin and ICAM-1. Briefly, ribonucleic acid was extracted from tissue samples using Trizol reagent (Invitrogen, Carlsbad, Calif.), and complementary DNA was prepared using Taqman reverse transcriptase reagents (Applied Biosystems, Foster City, Calif.). Primers for E-selectin and ICAM-1, respectively, were 59-GATGAAGCAAGTGCGTAT-39 and 39-GATGTAGGTTTCTGGGTT-59, and 59-AAACGGGAGATGAA2TGGT-39 and 39-ATGTGGATAATGGCGGTCT-59. The glyceraldehyde-3-phosphate dehydrogenase (GAPDH) gene (primers 59-GGCAAATTCAACGGCACAGT-39 and 39-CCCTTCGGGTAGTGGTAGA-59) was used as a reference to normalize E-selectin and ICAM-1 measurements.

RTPCR amplifications were conducted with the Absolute Blue SYBR Green/ROX kit (Thermo Scientific, Waltham, Mass.) on an ABI Prism 7000 sequence detection system (Applied Biosystems). E-selectin and ICAM-1 expression were calculated by the DDCt method and normalized to GAPDH expression in the same sample. The calculated values were expressed as fold increase over nonischemic myocardium.

### EXAMPLE X

### Statistical Analysis

The results are expressed as mean 6 SD. Data were compared with two-tailed Student t-tests. Statistical significance was defined as p<0.05. The relationship between the size of the risk area and the size of the region of postischemic contrast enhancement was assessed using linear regression analysis.

### EXAMPLE XI

### Molecular Imaging with Targeted Microbubbles(Mal) in an Inflammation Model

### Animal Procedure

Wistar Rats were intraperitoneally injected with lipopolysaccharide (LPS) in normal saline (dose: 1 mg/kg) to induce systemic inflammation. Four hours post-injection, rats were anesthetized and intubated.

### Ultrasound Molecular Imaging

Each rat received intravenous injection of targeted MB Esel(MAL) and control MB IgG in random order (2×10⁷ MB in 200 μL saline followed by a 200 μL saline flush). Baseline contrast ultrasound images were obtained before each microbubble injection and intermittent contrast ultrasound imaging were performed for 20 min to monitor the adhesion dynamics of MB in the myocardium. Acuson Sequoia 512 clinical ultrasound system (Siemens, Mountain View, Calif.) with a 15 L8 linear array transducer was used for imaging in CPS mode at 7 MHz.

### Image Analysis

Microbubble (MB) signals in the anterior myocardium and in the left ventricular cavity were also analyzed. Mean video intensity was calculated and displayed in dB scale. Signal intensity was linearized and a two-phase washout model

Iₜᵢₛₛᵤₑ=A_{f}e_{-aft}+Aᵣe^{-art}

was used to calculate the amount of MB retention in the myocardium.

### RT-qPCR

LPS treated and untreated (i.e., baseline) hearts were harvested. RNA extraction and reverse transcriptase real-time quantitative polymerase chain reaction (RT-qPCR) for E-selectin, ICAM and glyceraldehyde 3-phosphate dehydrogenase (GAPDH, a house keeping gene) in the heart were performed. E-selectin and ICAM mRNA expression level were expressed as percent of GAPDH.

### References

1. Krieglstein C F, Granger D N. Adhesion molecules and their role in vascular disease. Am J Hypertens 2001; 14:44S-54S, doi: 10.1016/S0895-7061(01)02069-6.

2. Nakamori S, Kameyama M, Imaoka S, et al. Involvement of carbohydrate antigen sialyl Lewisx in colorectal cancer metastasis. Dis Colon Rectum 1997; 40:420-31, doi: 10.1007/BF02258386.

3. Villanueva F S, Lu E, Bowry S, et al. Myocardial ischemic memory imaging with molecular echocardiography. Circulation 2007; 115:345-52, doi: 10.1161/CIRCULATIONAHA.106.633917.

4. Cao H, Huang S, Cheng J, et al. Chemical preparation of sialyl Lewis x using an enzymatically synthesized sialoside building block. Carbohydr Res 2008; 343:2863-9, doi:10.1016/j.carres.2008.06.020.

5. Ravindranath N M, Nishimoto K, Chu K, Shuler C. Cell-surface expression of complement restriction factors and sialyl Lewis antigens in oral carcinoma: relevance to chemo-immunotherapy. Anticancer Res 2000; 20:21-6.

6. Ogiso M, Shogomori H, Hoshi M. Localization of LewisX, sialyl- LewisX and alpha-galactosyl epitopes on glycosphingolipids in lens tissues. Glycobiology 1998; 8:95-105, doi:10.1093/glycob/8.1.95.

7. Wei K, Crouse L, Weiss J, et al. Comparison of usefulness of dipyridamole stress myocardial contrast echocardiography to technetium-99 m sestamibi single-photon emission computed tomography for detection of coronary artery disease (PB 127 multicenter phase 2 trial results). Am J Cardiol 2003; 91:1293-8, doi:10.1016/S0002-9149(03)00316-3.

8. Main M L, Ehlgen A, Coggins T R, et al Pulmonary hemodynamic effects of dipyridamole infusion in patients with normal and elevated pulmonary artery systolic pressure receiving PB127. J Am Soc Echocardiogr 2006; 19:1038-44, doi:10.1016/j.echo.2006.03.006.

9. Villanueva F S, Gertz E W, Csikari M, et al. Detection of coronary artery stenosis with power Doppler imaging. Circulation 2001; 103: 2624-30, doi:10.1161/01.CIR.103.21.2624.

10. Ottoboni S, Short R E, Kerby M B, et al. Characterization of the in vitro adherence behavior of ultrasound responsive double-shelled microspheres targeted to cellular adhesion molecules. Contrast Media Mol Imaging 2006; 1:279-90, doi:10.1002/cmmi.115.

11. Martens C L, Cwirla S E, Lee R Y, et al. Peptides which bind to Eselectin and block neutrophil adhesion. J Biol Chem 1995; 270: 21129-36, doi:10.1074/jbc.270.36.21129.

12. Villanueva F S, Jankowski R J, Manaugh C, Wagner W R. Albumin microbubble adherence to human coronary endothelium: implications for assessment of endothelial function using myocardial contrast echocardiography. J Am Coll Cardiol 1997; 30:689-93, doi:10.1016/S0735-1097(97)00197-6.

13. Lindner J R, Song J, Xu F, et al. Noninvasive ultrasound imaging of inflammation using microbubbles targeted to activated leukocytes. Circulation 2000; 102:2745-50, doi:10.1161/01.CIR.102.22.2745.

14. Weller G E, Lu E, Csikari M M, et al. Ultrasound imaging of acute cardiac transplant rejection with microbubbles targeted to intercellular adhesion molecule-1. Circulation 2003; 108:218-24, doi:10.1161/01.CIR0.0000080287.74762.60.

15. Fishbein M C, Meerbaum S, Rit J, et al. Early phase acute myocardial infarct size quantification: validation of the triphenyl tetrazolium chloride tissue enzyme staining technique. Am Heart J 1981; 101:593-600, doi:10.1016/0002-8703(81)90226-X.

16. Kaufmann B A, Lewis C, Xie A, et al. Detection of recent myocardial ischaemia by molecular imaging of P-selectin with targeted contrast echocardiography. Eur Heart J 2007; 28:2011-7, doi:10.1093/eurheartj/ehm176.

17. Porter T R. Cardiovascular imaging of remote myocardial ischemia: detecting a molecular trace of evidence left behind. Circulation 2007; 115:292-3, doi:10.1161/CIRCULATIONAHA.106.675413.

18. Klibanov A L, Rychak J J, Yang W C, et al. Targeted ultrasound contrast agent for molecular imaging of inflammation in highshear flow. Contrast Media Mol Imaging 2006; 1:259-66, doi:10. 1002/cmmi 113.

19. Burt C W. Summary statistics for acute cardiac ischemia and chest pain visits to United States EDs, 1995-1996. Am J Emerg Med 1999; 17:552-9, doi:10.1016/S0735-6757(99)90195-X.

20. Brieger D, Eagle K A, Goodman S G, et al. Acute coronary syndromes without chest pain, an underdiagnosed and undertreated high-risk group: insights from the Global Registry of Acute Coronary Events Chest 2004; 126:461-9, doi:10.1378/chest.126.2.461.

21. Christenson J, Innes G, McKnight D, et al. Safety and efficiency of emergency department assessment of chest discomfort. CMAJ 2004; 170:1803-7, doi:10.1503/cmaj.1031315.

22. Achar S A, Kundu S, Norcross W A. Diagnosis of acute coronary syndrome. Am Fam Physician 2005; 72:119-26.

23. Tucker J F, Collins R A, Anderson A J, et al. Early diagnostic efficiency of cardiac troponin I and troponin T for acute myocardial infarction. Acad Emerg Med 1997; 4:13-21, doi:10. 1111/j.1553-2712.1997.tb03637.x.

24. Bremerich J, Buser P, Bongartz G, et al. Noninvasive stress testing of myocardial ischemia: comparison of GRE-MRI perfusion and wall motion analysis to 99 mTc-MIBI-SPECT, relation to coronary angiography. Eur Radiol 1997; 7:990-5, doi:10.1007/s003300050238.

25. Oudkerk M, Stillman A E, Halliburton S S, et al. Coronary artery calcium screening: current status and recommendations from the European Society of Cardiac Radiology and North American Society for Cardiovascular Imaging. Eur Radiol 2008; 18:2785-807, doi:10.1007/s00330-008-1095-6.

26. Ruiz M, Takehana K, Petruzella F D, et al. Arbutamine stress perfusion imaging in dogs with critical coronary artery stenoses: (99 m)Tc-sestamibi versus (201)T1. J Nucl Med 2002; 43:664-70.

27. Vanhaecke J, Flameng W, Borgers M, et al. Evidence for decreased coronary flow reserve in viable postischemic myocardium. Circ Res 1990; 67:1201-10, doi:10.1161/01.RES.67.5.1201.

28. Villanueva F S, Glasheen W P, Sklenar J, Kaul S. Characterization of spatial patterns of flow within the reperfused myocardium by myocardial contrast echocardiography. Implications in determining extent of myocardial salvage. Circulation 1993; 88:2596-606, doi:10.1161/01.CIR.88.6.2596.

29. Dilsizian V, Bateman T M, Bergmann S R et al. Metabolic imaging with beta-methyl-p-[(123)I]-iodophenyl-pentadecanoic acid identifies ischemic memory after demand ischemia. Circulation 2005; 112:2169-74, doi:10.1161/CIRCULATIONAHA.104.530428.

30. Walzog B, Gaehtgens P. Adhesion molecules: the path to a new understanding of acute inflammation. News Physiol Sci 2000; 15: 107-13.

31. Scholz D, Devaux B, Hirche A, et al. Expression of adhesion molecules is specific and time-dependent in cytokine-stimulated endothelial cells in culture. Cell Tissue Res 1996; 284:415-23, doi:10.1007/s004410050602.

32. Weyrich A S, Buerke M, Albertine K H, Lefer A M. Time course of coronary vascular endothelial adhesion molecule expression during reperfusion of the ischemic feline myocardium. J Leukoc Biol 1995; 57:45-55.

33. Davidson B P, Kaufmann B A, Belcik J T, et al. Detection of antecedent myocardial ischemia with multiselectin molecular imaging. J Am Coll Cardiol 2012; 60:1690-7, doi:10.1016/j.jacc. 2012.07.027.

34. Fries J W, Williams A J, Atkins R C, et al. Expression of VCAM-1 and E-selectin in an in vivo model of endothelial activation. Am J Pathol 1993; 143:725-37.

I claim:

1. A composition comprising a dual shell polymer microbubble conjugated to a peptide having a selective affinity for an adhesion molecule.

2. The composition of claim 1, wherein said adhesion molecule is selected from the group consisting of E-selectin, P-selectin, ICAM, NCAM and VCAM.

3. The composition of claim 1, wherein said microbubble comprises an outer layer comprising a biologically compatible material.

4. The composition of claim 3, wherein said biologically compatible material is an amphiphlic material.

5. The composition of claim 1, wherein said microbubble comprises an inner layer comprising a biodegradable polymer.

6. The composition of claim 5, wherein said biodegradable polymer is a synthetic polymer.

7. The composition of claim 1, said microbubble comprises a hollow core.

8. The composition of claim 7, wherein said hollow core encapsulates an acoustically active gas.

9. A method comprising;
a) providing:
i) a pharmaceutically acceptable formulation comprising a dual shell polymer microbubble conjugated to a peptide having selective affinity for an adhesion molecule; and
ii) an ultrasound imaging device;
b) administering said formulation to a patient comprising at least one tissue overexpressing said adhesion molecule;
c) imaging said at least one tissue with said ultrasound imaging device;
d) measuring retention of said microbubble by said at least one tissue; and
e) diagnosing said patient with a medical condition when said patient microbubble retention is greater than a control microbubble retention.

10. The method of claim 9, wherein said at least one tissue is a cardiovascular tissue.

11. The method of claim 10, wherein said medical condition is a cardiovascular disease.

12. The method of claim 11, wherein said cardiovascular disease comprises a myocardial infarction.

13. The method of claim 9, wherein said microbubble retention comprises an interaction between said overexpressed adhesion molecule and said peptide having selective affinity for said adhesion molecule.


SEQUENCE LISTING (24 sequences)

SEQ ID NO: 1 - protein, 12 aa, Artificial Sequence
  Synthetic
DITWDQLWDLMK

SEQ ID NO: 2 - protein, 7 aa, Artificial Sequence
  Synthetic
IELLQAR

SEQ ID NO: 3 - protein, 12 aa, Artificial Sequence
  Synthetic
DITWDQLWDLMK

SEQ ID NO: 4 - protein, 13 aa, Artificial Sequence
  Synthetic
NDITWDQLWDLMK

SEQ ID NO: 5 - protein, 13 aa, Artificial Sequence
  Synthetic
NWKLDTLDMIWQD

SEQ ID NO: 6 - DNA, 18 bp, Artificial Sequence
  Synthetic
gatgaagcaagtgcgtat

SEQ ID NO: 7 - DNA, 18 bp, Artificial Sequence
  Synthetic
gatgtaggtttctgggtt

SEQ ID NO: 8 - DNA, 18 bp, Artificial Sequence
  Synthetic
aaacgggagatgaatggt

SEQ ID NO: 9 - DNA, 19 bp, Artificial Sequence
  Synthetic
atgtggataatggcggtct

SEQ ID NO: 10 - DNA, 20 bp, Artificial Sequence
  Synthetic
ggcaaattcaacggcacagt

SEQ ID NO: 11 - DNA, 19 bp, Artificial Sequence
  Synthetic
cccttcgggtagtggtaga

SEQ ID NO: 12 - protein, 13 aa, Artificial Sequence
  Synthetic
CDITWDQLWDLMK

SEQ ID NO: 13 - protein, 12 aa, Artificial Sequence
  Synthetic
DITWDQLWDLMK

SEQ ID NO: 14 - protein, 12 aa, Artificial Sequence
  Synthetic
DITWDELWKIMN

SEQ ID NO: 15 - protein, 12 aa, Artificial Sequence
  Synthetic
DYTWFELWDMMQ

SEQ ID NO: 16 - protein, 12 aa, Artificial Sequence
  Synthetic
QITWAQLWNMMK

SEQ ID NO: 17 - protein, 12 aa, Artificial Sequence
  Synthetic
DMTWHDLWTLMS

SEQ ID NO: 18 - protein, 12 aa, Artificial Sequence
  Synthetic
DYSWHDLWEMMS

SEQ ID NO: 19 - protein, 12 aa, Artificial Sequence
  Synthetic
EITWDQLWEVMN

SEQ ID NO: 20 - protein, 12 aa, Artificial Sequence
  Synthetic
HVSWEQLWDIMN

SEQ ID NO: 21 - protein, 12 aa, Artificial Sequence
  Synthetic
HITWDQLWRIMT

SEQ ID NO: 22 - protein, 12 aa, Artificial Sequence
  Synthetic
HITWDQLWNVMN

SEQ ID NO: 23 - protein, 12 aa, Artificial Sequence
  Synthetic
DISWDDLWIMMN

SEQ ID NO: 24 - protein, 12 aa, Artificial Sequence
  Synthetic
QITWDQLWDLMY